(19) United States
(12) Patent Application Publication
(10) Pub. No.: US 2009/0269339 A1
(43) Pub. Date: Oct. 29, 2009

(54) RESPONSES TO IMMUNIZATIONS IN RHEUMATOID ARTHRITIS PATIENTS TREATED WITH A CD20 ANTIBODY

(75) Inventors: Ariella Kelman, Hillsborough, CA (US); Benjamin L. Trzaskoma, San Mateo, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/431,057

(22) Filed: Apr. 28, 2009

Related U.S. Application Data
(60) Provisional application No. 61/048,874, filed on Apr. 29, 2008.

(51) Int. Cl. A61K 39/395

(52) U.S. Cl. 424/133.1; 424/172.1

(57) ABSTRACT

The present invention provides clinical data evaluating the efficacy of responses to immunizations in rheumatoid arthritis (RA) patients treated with a CD20 antibody.

### CROSS REFERENCE TO RELATED APPLICATION

This non-provisional application filed under 37 CFR § 1.53(b), claims the benefit under 35 USC § 119(e) of U.S. Provisional Application Ser. No. 61/048,874 filed on 29 Apr. 2008, which is incorporated by reference in entirety.

### FIELD OF THE INVENTION

The present invention provides clinical data evaluating the efficacy of responses to immunizations in rheumatoid arthritis (RA) patients treated with a CD20 antibody.

### BACKGROUND OF THE INVENTION

The CD20 antigen (also called human B-lymphocyte-restricted differentiation antigen, Bp35, or B1) is a four-pass, glycosylated integral membrane protein with a molecular weight of approximately 35 kD located on pre-B and mature B lymphocytes. The antigen is also expressed on greater than 90% of B-cell non-Hodgkin's lymphomas (NHL), but is not found on hematopoietic stem cells, pro-B cells, normal plasma cells, or other normal tissues. CD20 regulates early step(s) in the activation process for cell-cycle initiation and differentiation, and possibly functions as a calcium-ion channel. Undergoing phosphorylation in activated B cells, CD20 appears on the surface of B-lymphocytes at the pre-B-cell stage and is found on mature and memory B cells, but not plasma cells. CD20 has calcium-channel activity and may have a role in the development of B cells.

The rituximab (RITUXAN®) antibody is a genetically engineered chimeric murine/human monoclonal antibody directed against the CD20 antigen. Rituximab is the antibody called “C2B8” in U.S. Pat. No. 5,736,137 (Anderson et al.). Rituximab is indicated for the treatment of patients with relapsed or refractory low-grade or follicular, CD20-positive, B-cell NHL. In vitro mechanism-of-action studies have demonstrated that rituximab binds human complement and lyses lymphoid B-cell lines through CDC. Additionally, it has significant activity in assays for ADCC. Rituximab has been shown to have anti-proliferative effects in tritiated thymidine-incorporation assays and to induce apoptosis directly, while other anti-CD19 and CD20 antibodies do not. Rituximab sensitizes drug-resistant human B-cell lymphoma cell lines to the cytotoxic effects of doxorubicin and other toxins. In vivo preclinical studies have shown that rituximab depletes B cells from the peripheral blood, lymph nodes, and bone marrow of cynomolgus monkeys.

Rituximab was approved in the U.S. in November 1997 for the treatment of patients with relapsed or refractory low-grade or follicular CD20⁺ B-cell NHL at a dose of 375 mg/m² weekly for four doses. In April 2001, rituximab was additionally approved in the U.S. for treating low-grade NHL: re-treatment (weekly for four doses) and an additional dosing regimen (weekly for eight doses). Since approval, patients have been exposed to rituximab either as monotherapy or in combination with immunosuppressant or chemotherapeutic drugs. Patients have also been treated with rituximab as maintenance therapy for up to two years. Rituximab has been used in the treatment of malignant and nonmalignant plasma cell disorders.

Other CD20 antibodies include, e.g, the ⁹⁰Y-labeled 2B8 murine antibody designated “Y2B8” (ZEVALIN®) (Biogen-Idec, Inc.) (e.g., U.S. Pat. No. 5,736,137, Anderson et al.; ATCC deposit HB11388); murine IgG2a “B1” or “tositumomab,” optionally labeled with ¹³¹I to produce the “131I-B1” or “iodine I131 tositumomab” antibody (BEXXAR™) (Corixa; Coulter Pharmaceutical, Inc.) (e.g., U.S. Pat. No. 5,595,721, Kaminski et al.); murine monoclonal antibody “1F5” (e.g., Press et al. Blood, 69(2):584-591 (1987) and its variants, e.g., “framework patched” or humanized 1F5 (e.g., WO 2003/002607, Leung; ATCC deposit HB-96450); murine and chimeric 2H7 antibody (e.g., U.S. Pat. No. 5,677,180, Robinson et al.); humanized 2H7 antibodies such as rhuMAb2H7 and other versions (Genentech, Inc.) (e.g., WO 2004/056312, Adams et al., and other references noted below); the human antibody targeted at CD20 called 2F2, HUMAX-CD20™, or ofatumumab (GlaxoSmithKline; GenMab A/S) (e.g., Glennie and van de Winkel, Drug Discovery Today, 8:503-510 (2003); Cragg et al., Blood, 101: 1045-1052 (2003); and US 2004/0167319, Teeling et al.); human monoclonal antibodies against CD20 (GenMab A/S/Medarex, Inc.) (e.g., WO 2004/035607 and WO 2005/103081, Teeling et al.); antibodies to CD20 having complex N-glycoside-linked sugar chains bound to the Fc region (Kyowa Hakko) (e.g., US 2004/0093621, Shitara et al.); a chimerized or humanized monoclonal antibody binding to an extracellular epitope of CD20 (Biomedics Inc.) (e.g., WO 2006/106959, Numazaki et al.); monoclonal antibodies and fragments binding to CD20 (e.g., WO 2005/000901, Tedder et al.) such as HB20-3, HB20-4, HB20-25, and MB20-11; small, modular immunopharmaceuticals (SMIPs) binding to CD20 (Wyeth, Trubion Pharmaceuticals, Inc.), including TRU-015 (e.g., US 2005/0186216; US 2005/0202534; US 2005/0202028; US 2005/136049; and US 2005/0202023, Ledbetter et al., and US 2007/0059306, Grosmaire et al.); CD20-binding antibodies including the AME series of antibodies (Eli Lilly and Co., Applied Molecular Evolution, Inc.), such as AME 33 (e.g., US 2005/0025764, Watkins et al.) and AME 133 and AME 133v antibodies (e.g., US 2005/0136044, Watkins and Pancook) (see also, e.g., WO 2004/103404 and US 2006/0251652, Watkins et al.) and the CD20 antibodies with Fc mutations (e.g., WO 2005/070963, Allan et al.); CD20-binding molecules such as those set forth in WO 2005/016969 and US 2005/0069545, Carr et al.); bispecific antibodies set forth in WO 2005/014618 (Chang et al.); humanized LL2 and similar antibodies (Immunomedics, Inc.) (e.g., U.S. Pat. No. 7,151,164 and US 2005/0106108, Hansen); A20 antibodies (Immunomedics, Inc.) such as chimeric A20 (cA20) or humanized A20 antibody (hA20, IMMUN-106™, veltuzumab) (e.g., US 2003/0219433, Hansen et al.); fully human antibodies against CD20 (Amgen/AstraZeneca) (e.g., WO 2006/130458, Gazit et al.); antibodies against CD20 (Avestha Gengraine Technologies Pvt Ltd.) (e.g., WO 2006/126069, Morawala); chimeric or humanized B-Ly1 antibodies to CD20 (Roche/GlycArt Biotechnology AG) such as GA101 (e.g., WO 2005/044859; US 2005/0123546; US 2004/0072290; and US 2003/0175884, Umana et al.); and monoclonal antibodies L27, G28-2, 93-1B3, B-C1, or NU-B2 available from the International Leukocyte Typing Workshop (e.g., Valentine et al., In: Leukocyte Typing III (McMichael, Ed., p. 440, Oxford University Press (1987)). This list provides representative CD20 antibodies, but is not exhaustive.

RA is a debilitating autoimmune disease that affects more than two million Americans and hinders the daily activities of sufferers. The damage that occurs in RA is a result of the immune system attacking joint tissue, causing painful chronic inflammation, irreversible destruction of cartilage, tendons and bones, which often results in disability. Common RA symptoms include inflammation of the joints, swelling, fatigue, stiffness and pain. Additionally, since RA is a systemic disease, it can have effects in other tissues such as the lungs and eyes.

Earlier studies of rituximab in RA include a Phase II study (WA16291) conducted in patients with RA, providing 48-week follow-up data on safety and efficacy of rituximab. Edwards et al. N. Eng. J. Med. 350(25): 2572-2581 (2004). Patients were evenly randomized to four treatment arms: methotrexate, rituximab alone, rituximab plus methotrexate, and rituximab plus cyclophosphamide. The treatment regimen of rituximab was one gram administered intravenously on days 1 and 15. Infusions of rituximab were well tolerated by most RA patients, 36% of whom experienced at least one adverse event during their first infusion (compared with 30% of patients receiving placebo). Overall, the majority of adverse events was considered to be mild to moderate in severity and was well balanced across all treatment groups. Nineteen total serious adverse events occurred across the four arms over the 48 weeks, which were slightly more frequent in the rituximab/cyclophosphamide group. The incidence of infections was well balanced across all groups. The mean rate of serious infection in this RA patient population was 4.66 per 100 patient-years, which is lower than the rate of infections requiring hospital admission in RA patients (9.57 per 100 patient-years) reported in a community-based epidemiologic study.

The DANCER Phase IIb trial evaluated the efficacy of rituximab and methotrexate in disease-modifying anti-rheumatic drug (DMARD)-resistant RA patients, with rituximab given at doses of 500 mg or 1000 mg at days 1 and 15. The ACR responses for both doses of rituximab were statistically superior to placebo at 6 months. No difference between the two rituximab doses was seen, and analysis of the utility of the oral corticosteroids revealed no significant impact on ACR response. Emery et al Arthritis and Rheumatism 54:1390-400 (2006).

The REFLEX Phase III trial evaluated the efficacy of rituximab and methotrexate in RA patients with an inadequate response to anti-TNF-alpha therapy, with rituximab given at a dose of 1000 mg. Patients treated with rituximab under the trial conditions had demonstrated improvements in the signs and symptoms of active disease, with a significant benefit over six months. Cohen et al. Arthritis and Rheumatism 54:2793-2806 (2006).

Elderly individuals (≧65 years) generally mount a poor humoral immune response to vaccines such as influenza and tetanus toxoid (Burns et al. J Gerontol 48(6):B231-6 (1993)). One month after an influenza vaccine, only half of the elderly subjects in a clinical trial exhibited an intact humoral response, and only a third of subjects had an intact cell-mediated response (Rastogi et al. Clin Diagn Lab Immunol 2(1): 120-1 (1995)), although the time to peak serum antibody response to influenza vaccine in elderly subjects has been observed to be similar to that of younger individuals (Bernstein et al. Vaccine 17(1):82-4 (1999)). Additionally, a study to evaluate immune response over time showed that antibody and T-cell proliferative responses to influenza vaccine in the elderly were both significantly and consistently lower than responses in younger individuals (Murasko et al. Exp Gerontol 37(2-3):427-39 (2002)).

In a study evaluating methotrexate (MTX) use on vaccine responses in subjects with psoriatic arthritis (Mease et al. Arthritis Rheumatism 44(Suppl 9):S91 (2001)), variables associated with a higher risk of poor response to vaccinations included MTX use, concomitant diabetes, age >40 years, and female sex. In another study evaluating subjects with RA, immune responses to pneumococcal polysaccharide vaccine were significantly decreased in subjects treated with MTX and the effect of MTX was greatest for subjects >60 years (O'Dell et al. Arthritis Rheum 35 (Suppl 9):S197 (1992)).

RA patients have lower responses to vaccines and skin tests versus healthy controls. See, Elkayam et al., Seminars in Arthritis and Rheumatism 33(4):283-288 (2004), Kapetanovic et al., Rheumatology 46:608-611 (2007), Ravikumar et al., Current Rheumatology Reports 9:407-415 (2007), and Emery et al., Annals of the Rheumatic Diseases 43:430-434 (1984). Predictors of decreased response include MTX use and older age. See, Mease et al., J. Rheumatol. 31:1356-1361 (2004), and O'Dell et al. Arthritis Rheum 35 (Suppl 9):S197 (1992). Most vaccine studies in RA patients are small and either uncontrolled or use healthy controls. Two large placebo-controlled vaccine trials have evaluated vaccine responses in inflammatory arthritis patients treated with anti-TNF agents. Kaine et al. J. Rheumatol. 34:272-279 (2007) (RA), and Mease et al., J. Rheumatol. 31:1356-1361 (2004) (psoriatic arthritis). These trials showed generally preserved responses to pneumococcal polysaccharide antigens with use of anti-TNF agents for 1-2 months.

Preclinical studies have evaluated vaccination responses with rituximab. Gonzales-Stawinski et al. Clin Immunol 98(2): 175-9 (2001) found that baboons treated with rituximab and dinitrobenzene couped to keyhole limpet hemocyanin (DNP-KLH) vaccine displayed both decreased primary and memory response. In another study (Schmitz et al. J Virol 77:2165-73 (2003)), rhesus moneys treated with rituximab had a decreased humoral response to tetanus toxoid vaccine. DiLillo et al. studied rituximab and DNP-KLH in mice, and observed decreased primary and memory responses. DiLillo et al., J. Immunol. 180:361-371 (2008).

Clinical studies of response to vaccine with rituximab are summarized in the following table:

| Study | Disease and Sample Size | Vaccine(s) | Response |
|---|---|---|---|
| Van der Kolk et al. | Relapsed, low grade | KLH, HAV | Failed primary |
| Blood 100: 2257-2259 | Lymphoma | Tetanus, Polio | response, decreased |
| (2002) | N = 11 | | memory responses |
| | | | after Rituximab |
| Horwitz et al. Blood | Aggressive Non-Hodgkin's | Tetanus, hemophilus | Decreased T-cell |
| 103: 777-183 (2004) | lymphoma (Rituximab + | influenza (conjugate), | independent response |
| | autologous hematopoietic | pneumococcal | (to pneumococcal |
| | cell transplantation) | polysaccharide | polysacharide); No |
| | N = 22 | | decrease in T-cell |
| | | | dependent responses |
| | | | (tetanus, H. influenza) |
| Bearden et al. Am. J. | Chronic Renal Failure | PhiX174 | Decreased primary |
| Transplantation 5: | N = 18 | | and memory response |
| 50-57 (2005) | | | |
| Oren et al., Ann. | Rheumatoid Arthritis, | Influenza | Rituximab group |
| Rheum. Dis | N = 64; RA standard of care | | showed vaccine |
| (Published online | (SOC) N = 29, RA | | response for 2 out of |
| Dec. 4, 2007 | Rituximab N = 14; Controls | | 3 antigens |
| doi: 10.1136/ard.2007. | N = 21 | | |
| 077461) pp. 1-19 (2007). | | | |
| Albert et al., Ann. | Systemic Lupus | Tetanus | Most failed responses |
| Rheum. Dis. | N = 14 | 23VPPV | to both 7 months after |
| (Published online | | | Rituximab |
| Feb. 4, 2008 | | | |
| doi: 10.1136/ard.2007. | | | |
| 083162) pp. 1-28 | | | |
| (2008). | | | |

In the randomized Phase II trial (Study WA16291), small decreases in mean serum levels of immunoglobulin were observed in the rituximab groups and, to a lesser extent, in the MTX group. At Week 24, mean and median serum levels of immunoglobulin (total Ig) and Ig isotypes (A, G, and M) showed small decreases from baseline in the rituximab arms and, to a lesser extent, in the control arm. Decreases were greatest in the combination treatment arms. Despite these decreases, mean and median values remained well within the normal ranges. However, there was no clear difference between treatment groups in mean serum levels of anti-tetanus antibody during the study. In the Rituximab+MTX group, the mean change in anti-tetanus antibody titer at 24 weeks was −0.1±0.6 compared with 0.0±0.6 in the MTX alone group. Edwards et al. N. Eng. J. Med. 350(25): 2572-2581 (2004).

In the Phase IIb Study WA17043/U2644g, Rituximab did not appear to significantly alter the levels of antibody titers for mumps, rubella, varicella, tetanus, influenza, and pneumococcus. Immunoglobulins IgG, IgA, and IgM were decreased at Week 24 in all groups that received active treatment. Despite these decreases, mean values remained well within the normal range for total and individual Ig isotype. Emery et al Arthritis and Rheumatism 54:1390-400 (2006).

In the Phase III study (WA 17042), levels of IgG, IgA, and IgM were decreased at Week 24 in both the MTX alone group and in the active Rituximab+MTX group. Individual isotypes and total immunoglobulin levels were decreased in the active group compared with the control group, but mean values remained within the normal range. Cohen et al. Arthritis and Rheumatism 54:2793-2806 (2006).

Other publications reporting the effect of Rituximab on pre-existing antibody levels include: Cambridge et al. Arth. Rheum. 54: 3612-22 (2006) in systemic lupus erythematosus (SLE); Cambridge et al. Arth. Rheum. 54: 723-32 (2006) in RA; and Vallerskog et al. Clin. Immunol. 122: 62-74 (2007) in systemic lupus erythematosus (SLE).

Hassan et al. Clin. Can. Res. 10: 16-18 (2004) found that pretreatment with Rituximab did not inhibit the human immune response against the immunotoxin, LMB-1.

### SUMMARY OF THE INVENTION

The present application provides clinical data evaluating the effects of CD20 antibody on immune responses in human RA patients. The primary objective of this study was to characterize the immune response to a protein vaccine—tetanus toxoid vaccine—in RA patients treated with rituximab in combination with methotrexate (MTX) (Active group) compared with that of subjects treated with MTX alone (Control group). The secondary objectives of this study were to characterize the immune responses in Active and Control groups to: the 23-valent pneumococcal polysaccharide vaccine; keyhole limpet hemocyanin (KLH); and the delayed-type hypersensitivity (DTH) response to Candida albicans.

A tetanus toxoid adsorbed vaccine was administered to assess whether rituximab affects antibody production to an antigen that the body has an existing immunity to prior to treatment. A 23-valent pneumococcal polysaccharide vaccine was selected to provide an additional measure for a clinically relevant antigen unknown to the majority of individuals. KLH was used to test primary humoral response as it is a novel immunogen for most individuals. Responses to intradermal skin testing with Candida albicans antigens were evaluated to measure T-cell memory.

Accordingly, in a first aspect, the invention concerns a method of treating a human rheumatoid arthritis (RA) patient comprising administering to the patient: (a) a CD20 antibody in an amount effective to treat the RA, and (b) a protein vaccine in an amount effective to mount a memory immune response to the protein vaccine. The memory immune response was observed in the example herein, in spite of the fact that the RA patient was still B-cell depleted (due to administration of the CD20 antibody) at the time of vaccination. Preferably the protein vaccine is a tetanus toxoid vaccine. Generally, the protein vaccine is administered to the patient following administration of the CD20 antibody, e.g. from about one month to about twelve months after administration of the CD20 antibody, most preferably about six months after administration of the CD20 antibody.

The invention further concerns a method of mounting a 4-fold increase in anti-protein titer in a human rheumatoid arthritis (RA) patient following vaccination with a protein vaccine, comprising administering the protein vaccine to a RA patient who has been treated with a CD20 antibody, and measuring the 4-fold increase in anti-protein titer.

Additionally, the invention provides a method of treating human patients having rheumatoid arthritis (RA) comprising treating a first group of the RA patients with a CD20 antibody, methotrexate, and a protein vaccine, and treating a second group of the RA patients with methotrexate and the vaccine but not the CD20 antibody, and determining that memory immune responses raised by the first and second groups of patients are about the same.

In another aspect, the invention concerns a method for advertising a CD20 antibody comprising promoting the use of the CD20 antibody for treating a human rheumatoid arthritis (RA) patient, wherein the RA patient is to mount an effective memory immune response to a protein vaccine administered following administration of the CD20 antibody.

Also provided is a method of providing a pharmaceutical composition for treating a human rheumatoid arthritis (RA) patient, comprising combining a container holding a pharmaceutically acceptable composition comprising a CD20 antibody with a package insert, wherein the package insert promotes the use of the composition to treat a RA patient who is able to mount an effective memory immune response to a protein vaccine administered following administration of the CD20 antibody.

### BRIEF DESCRIPTION OF THE DRAWINGS

FIG. 1 provides an overview of the study design in the example.

FIG. 2 summarizes specific antigens tested.

FIG. 3 summarizes positive responses to 23-valent pneumococcal polysaccharide vaccine.

FIG. 4 summarizes positive responses to at least 1, 2, 3, 4, 5, or 6 of 12 pneumococcal antibody serotypes.

### DETAILED DESCRIPTION OF THE PREFERRED EMBODIMENTS

### I. Definitions

A “B cell” is a lymphocyte that matures within the bone marrow, and includes a naïve B cell, memory B cell, or effector B cell (plasma cell). The B cell herein is a normal or non-malignant B cell.

For the purposes herein, the “human CD20” antigen, or “human CD20,” is an about 35-kDa, non-glycosylated phosphoprotein found on the surface of greater than 90% of B cells from peripheral blood or lymphoid organs in humans. CD20 is present on both normal B cells as well as malignant B cells, but is not expressed on stem cells. Other names for CD20 in the literature include “B-lymphocyte-restricted antigen” and “Bp35.” The CD20 antigen is described in Clark et al., Proc. Natl. Acad. Sci. (USA), 82:1766 (1985), for example.

For the purposes herein, a “vaccine” is a substance or group of substances used to cause the immune system of a subject or patient to respond to a tumor or a microorganism, such as a bacteria or virus. Generally such vaccine will comprise one or more antigen(s) and, optionally, pharmaceutically acceptable carrier(s) and/or adjuvant(s).

An “antigen’ is a compound or composition which is able to elicit or mount an immune response in subject or patient vaccinated therewith.

An “adjuvant” herein is a vehicle or agent used to enhance antigenicity. Examples include a suspension of minerals (e.g. alum, aluminum hydroxide or phosphate) on which antigen is adsorbed, or water-in-oil emulsion in which antigen solution is emulsified in mineral oil (e.g. Freund's adjuvant).

A “protein vaccine” is one comprising at least one protein as an antigen used to stimulate an immune response thereagainst in a subject or patient. Examples include: tetanus toxoid vaccine, influenza, and protein conjugate vaccines such as protein conjugate pneumococcal vaccines, and H. influenzae protein conjugate vaccines.

A “polysaccharide vaccine” is a vaccine comprising at least one polysaccharide as an antigen used to generate an immune response thereagainst in a subject or patient. Examples include pneumococcal polysaccharide vaccine, memingococcal polysaccharide vaccine, and polysaccharide vaccine of Salmonella typhi.

A “recall antigen” or “memory antigen” is an antigen that a subject or patient has been previously vaccinated with or exposed to. Exemplary such antigens include tetanus toxoid. Such antigens can elicit a recall or memory immune response in a subject or patient.

The expression “neoantigen” refers to an antigen that a subject or patient vaccinated therewith has not previously been exposed to, or vaccinated with. Examples include pneumococcal polysaccharide vaccine (e.g. PNEUMOVAX®) (at least parts thereof in at least some individuals), Keyhole Limpet Hemocyanin (KLH), HepA, PhiX174, rabies vaccine, hepatitis A vaccine, hepatitis B vaccine, Varicella vaccine, etc. Such antigens may elicit a primary humoral response in a subject or patient.

The term “humoral response” is used to describe an immune response against foreign antigen(s) that is mediated by antibodies produced by B-cells.

A “primary humoral response” results from the activation of naive lymphocytes (B cells). A primary response to antigen is generally characterized by a lag time, which is the period of time from antigen encounter until the production of plasma cells and memory cells.

The expression “recall response” or “memory response” refer to the immune response to subsequent administration of an antigen.

The term “antibody” herein is used in the broadest sense and specifically covers monoclonal antibodies, polyclonal antibodies, multispecific antibodies (e.g. bispecific antibodies) formed from at least two intact antibodies, and antibody fragments, so long as they exhibit the desired biological activity.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The “variable region” or “variable domain” of an antibody refers to the amino-terminal domain of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a β-sheet configuration, connected by three HVRs, which form loops connecting, and in some cases forming part of, the β-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in ADCC.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W. B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment that contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The “Fab” fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain, including one or more cysteines from the antibody-hinge region. Fab′-SH is the designation herein for Fab′, in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains that enables the scFv to form the desired structure for antigen binding. For a review of scFv, see, e.g., Pluckthün, in The Pharmacology of Monoclonal Antibodies, vol. 113, Rosenburg and Moore eds. (Springer-Verlag, New York: 1994), pp 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med., 9:129-134 (2003); and Hollinger et al., Proc. Natl. Acad. Sci. USA, 90: 6444-6448 (1993). Triabodies and tetrabodies are also described in Hudson et al., Nat. Med., 9:129-134 (2003).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas, 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol., 222: 581-597 (1992); Sidhu et al., J. Mol. Biol., 338(2): 299-310 (2004); Lee et al., J. Mol. Biol., 340(5): 1073-1093 (2004); Fellouse, Proc. Natl. Acad. Sci. USA, 101(34): 12467-12472 (2004); and Lee et al., J. Immunol. Methods, 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90: 2551 (1993); Jakobovits et al., Nature, 362: 255-258 (1993); Bruggemann et al., Year in Immunol., 7:33 (1993); U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnol., 14: 845-851 (1996); Neuberger, Nature Biotechnol., 14: 826 (1996); and Lonberg and Huszar, Intern. Rev. Immunol., 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g., U.S. Pat. No. 4,816,567 and Morrison et al., Proc. Natl. Acad. Sci. USA, 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all, or substantially all, of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature, 321:522-525 (1986); Riechmann et al., Nature, 332:323-329 (1988); and Presta, Curr. Op. Struct. Biol., 2:593-596 (1992). See also, for example, Vaswani and Hamilton, Ann. Allergy, Asthma & Immunol., 1:105-115 (1998); Harris, Biochem. Soc. Transactions, 23:1035-1038 (1995); Hurle and Gross, Curr. Op. Biotech., 5:428-433 (1994); and U.S. Pat. Nos. 6,982,321 and 7,087,409.

A “human antibody” is one that possesses an amino-acid sequence that corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the r regions of an antibody-variable domain that are hypervariable in sequence and/or form structurally defined loops. Generally, antibodies comprise six HVRs; three in the VH(H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity, 13:37-45 (2000) and Johnson and Wu in Methods in Molecular Biology, 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature, 363:446-448 (1993) and Sheriff et al., Nature Struct. Biol., 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk, J. Mol. Biol., 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat Numbering) |
| H1 | H31-H35 | H26-H35 | H26-H32 | H30-H35 | (Chothia Numbering) |
| H2 | H50-H65 | H50-H58 | H53-H55 | H47-H58 | |
| H3 | H95-H102 | H95-H102 | H96-H101 | H93-H101 | |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino-acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat-numbered sequence.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology, 10:779-783 (1992) describes affinity maturation by VH- and VL-domain shuffling. Random mutagenesis of HVR and/or framework residues is described by, for example: Barbas et al., Proc Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154(7):3310-9 (1995); and Hawkins et al., J. Mol. Biol., 226:889-896 (1992).

“Growth-inhibitory” antibodies are those that prevent or reduce proliferation of a cell expressing an antigen to which the antibody binds. For example, the antibody may prevent or reduce proliferation of B cells in vitro and/or in vivo.

Antibodies that “induce apoptosis” are those that induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptic bodies).

Antibody “effector functions” refer to those biological activities attributable to the Fc region (a native-sequence Fc region or amino-acid-sequence-variant Fc region) of an antibody, and vary with the antibody isotype. Examples of antibody effector functions include: C1q binding and CDC; Fc-receptor binding; ADCC; phagocytosis; down-regulation of cell-surface receptors (e.g., B-cell receptor); and B-cell activation.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

A “functional Fc region” possesses an “effector function” of a native-sequence Fc region. Exemplary “effector functions” include C1q binding; CDC; Fc-receptor binding; ADCC; phagocytosis; down-regulation of cell-surface receptors (e.g., B-cell receptor), etc. Such effector functions generally require the Fc region to be combined with a binding domain (e.g. an antibody-variable domain) and can be assessed using various assays as disclosed, for example, in definitions herein.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, a composition comprising an antibody having an Fc region according to this invention can comprise an antibody with K447, with all K447 removed, or a mixture of antibodies with and without the K447 residue.

“Fc receptor” or “FcR” describes a receptor that binds to the Fc region of an antibody. In some embodiments, an FcR is a native-human FcR. In some embodiments, an FcR is one that binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see, e.g., Daëron, Annu. Rev. Immunol., 15:203-234 (1997)). FcRs are reviewed, for example, in Ravetch and Kinet, Annu. Rev. Immunol, 9:457-92 (1991); Capel et al., Immunomethods, 4:25-34 (1994); and de Haas et al., J. Lab. Clin. Med., 126:330-41 (1995).

The term “Fc receptor” or “FcR” also includes the neonatal receptor, FcRn, which is responsible for the transfer of maternal IgGs to the fetus (Guyer et al., J. Immunol., 117:587 (1976) and Kim et al., J. Immunol., 24:249 (1994)) and regulation of homeostasis of immunoglobulins. Methods of measuring binding to FcRn are known (see, e.g., Ghetie and Ward, Immunology Today, 18 (12):592-598 (1997); Ghetie et al., Nature Biotechnology, 15 (7):637-640 (1997); Hinton et al., J. Biol. Chem., 279(8):6213-6216 (2004); and WO 2004/92219 (Hinton et al.)).

Binding to human FcRn in vivo and serum half-life of human FcRn high-affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See, also, for example, Shields et al., J. Biol. Chem., 9(2): 6591-6604 (2001).

“Human effector cells” are leukocytes that express one or more FcRs and perform effector functions. In certain embodiments, the cells express at least FcγRIII and perform ADCC effector function(s). Examples of human leukocytes that mediate ADCC include peripheral blood mononuclear cells (PBMC), natural-killer (NK) cells, monocytes, cytotoxic T cells, and neutrophils. The effector cells may be isolated from a native source, e.g., from blood.

“Antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enables these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol., 9:457-492 (1991). To assess ADCC activity of a molecule of interest, an in vitro ADCC assay, such as that described in U.S. Pat. No. 5,500,362, 5,821,337 or 6,737,056 may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. USA, 95:652-656 (1998).

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods, 202:163 (1996), may be performed. Polypeptide variants with altered Fc-region amino acid sequences (polypeptides with a variant Fc region) and increased or decreased C1q binding capability are described, e.g., in U.S. Pat. No. 6,194,551 and WO 1999/51642. See, also, e.g., Idusogie et al., J. Immunol., 164: 4178-4184 (2000).

A “CD20 antibody” or “anti-CD20 antibody” herein refers to an antibody that comprises one or more antigen binding sites that bind the human CD20 antigen. These terms as used herein expressly include the various CD20 antibodies identified throughout the disclosure and others disclosed in the literature, but specifically include at least the following CD20 antibodies: (1) rituximab (RITUXAN®) further defined below, (2) humanized 2H7 antibodies as defined below, (3) ofatumumab (HUMAX-CD20™), an IgG1κ human MAb; (4) veltuzumab (IMMUN-106™ or hA20), a humanized engineered antibody with complementarity-determining regions (CDRs) of murine origin and with 90% of the human framework regions identical to epratuzumab (a humanized anti-CD22 IgG1 antibody); (5) a small, modular immunopharmaceutical (SMIP) (herein called immunopharmaceutical) (also known as TRU-015); (6) a CD20-binding molecule that is an antibody designated AME 33 or AME 133 or AME 133v (otherwise known as LY2469298), which binds with an increased affinity to the FcγRIIIa (CD16)); and (7) a humanized type II CD20 antibody of the isotype IgG1 with a glycoengineered Fc portion (bisected afucosylated carbohydrates in the Fc region) and a modified elbow hinge, known as GA101. All of these antibodies are further described below, including the full-length or variable-region sequences thereof and defining literature.

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof that retain the ability to bind CD20.

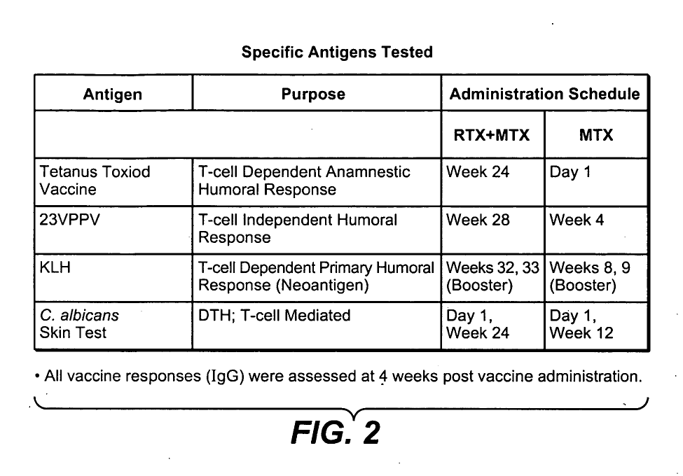

Purely for the purposes herein and unless indicated otherwise, “humanized 2H7 antibody” refers to a humanized CD20 antibody with the sequences provided immediately below and/or described in US 2006/0034835 and WO 2004/056312 (both Lowman et al.); US 2006/0188495 (Barron et al.); and US 2006/0246004 (Adams et al.). Briefly, humanization of the murine anti-human CD20 antibody, 2H7 (also referred to herein as m2H7, m for murine), was carried out in a series of site-directed mutagenesis steps. The murine 2H7 antibody variable region sequences and the chimeric 2H7 with the mouse V and human C have been described, e.g., in U.S. Pat. Nos. 5,846,818 and 6,204,023. The CDR residues of 2H7 were identified by comparing the amino acid sequence of the murine 2H7 variable domains (disclosed in U.S. Pat. No. 5,846,818) with the sequences of known antibodies (Kabat et al., Sequences of Proteins of Immunological Interest, Ed. 5 (Public Health Service, National Institutes of Health, Bethesda, Md., 1991)). The CDRs for the light and heavy chains were defined based on sequence hypervariability (Kabat et al., supra). With synthetic oligonucleotides, site-directed mutagenesis (Kunkel, Proc. Natl. Acad. Sci. USA, 82:488-492 (1985)) was used to introduce all six of the murine 2H7CDRs into a complete human Fab framework corresponding to a consensus sequence V_{κ}I, V_{H}III (V_{L} kappa subgroup I, V_{H} subgroup III) contained on plasmid pVX4 (see FIG. 2 in WO 2004/056312). Further modifications of the V regions (CDR and/or FR) were made in the phagemid pVX4 by site-directed mutagenesis. Plasmids for expression of full-length IgG's were constructed by subcloning the V_{L} and V_{H} domains of chimeric 2H7 Fab as well as humanized Fab versions 2 to 6 into previously described pRK vectors for mammalian cell expression (Gorman et al., DNA Prot. Eng. Tech., 2:3-10 (1990)).

The following 2H7 antibodies are included within the definition herein:

(1) A humanized antibody comprising the VL sequence:

(SEQ ID NO: 1)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKR;

and the VH sequence:

(SEQ ID NO: 2)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYEDVWGQGTLVTVSS.

(2) A humanized antibody comprising the VL sequence:

(SEQ ID NO: 3)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR;

and the VH sequence:

(SEQ ID NO: 4)

EVQLVESGGGLVQPGGSLRLSCAASGYTFTSYNMHWVRQAPGKGLEWVGA
IYPGNGAISYNQKFKGRFTISVDKSKNTLYLQMNSLRAEDTAVYYCARVV
YYSASYWYFDVWGQGTLVTVSS.

(3) A humanized antibody comprising the VL sequence:

(SEQ ID NO: 5)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR;

and the VH sequence:

(SEQ ID NO: 6)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYSYFDVWGQGTLVTVSS.

(4) A humanized antibody comprising a full-length light (L) chain having the sequence of SEQ ID NO:7, and a full-length heavy (H) chain having the sequence of one of SEQ ID NO:8, or SEQ ID NO:9, wherein the sequences are indicated below.
(5) A humanized antibody comprising a full-length light (L) chain having the sequence of SEQ ID NO:10, and a full-length heavy (H) chain having the sequence of one of SEQ ID NO:11, SEQ ID NO: 12, SEQ ID NO:13, SEQ ID NO:14, SEQ ID NO:15, or SEQ ID NO:16, wherein the sequences are indicated below.

SEQ ID NO: 7:
DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
SEQ ID NO: 8:
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEQ ID NO: 9:
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEQ ID NO: 10:
DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
SEQ ID NO: 11:
EVQLVESGGGLVQPGGSLRLSCAASGYTFTSYNMHWVRQAPGKGLEWVGA
IYPGNGATSYNQKEKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEO ID NO: 12:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCAVSNKALPAPIEATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEQ ID NO: 13:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEO ID NO: 14:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHWHYTQKSLSLSP
G
SEQ ID NO: 15:
EVQLVESGGGLVQPGGSLRLSCAASGYTFTSYNMHWVRQAPGKGLEWVGA
IYPGNGATSYNQKFKGRETISVDKSKNTLYLQMNSLRAEDTAVYYCARVV
YYSYRYWYFDVWGQGTLVTVSSASTKGPSVFPLAPSSKSTSGGTAALGCL
VKDYEPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVESCSVMHEALHNHYTQKSLSLSP
G
SEQ ID NO: 16:
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G

The murine anti-human CD20 antibody, m2H7 comprises the variable region sequences:

### VL Sequence:

(SEQ ID NO: 17)

QIVLSQSPAILSASPGEKVTMTCRASSSVSYMHWYQQKPGSSPKPWIYAP
SNLASGVPARFSGSGSGTSYSLTISRVEAEDAATYYCQQWSFNPPTFGAG
TKLELK

### VH Sequence:

(SEQ ID NO: 18)

QAYLQQSGAELVRPGASVKMSCKASGYTFTSYNMHWVKQTPRQGLEWIGA
IYPGNGDTSYNQKFKGKATLTVDKSSSTAYMQLSSLTSEDSAVYFCARVV
YYSNSYWYFDVWGTGTTVTVS

In the CD20 antibodies that comprise an Fc region, the C-terminal lysine (residue 447 according to the EU numbering system) of the Fc region may be removed, for example, during purification of the antibody or by recombinant engineering of the nucleic acid encoding the antibody polypeptide. For example, hA20 can comprise an Fc region including the K447 residue, or with all the K447 residues removed, or a mixture of antibodies having Fc regions with and without the K447 residue.

In certain embodiments, the CD20 antibody useful herein further comprises amino acid alterations in the IgG Fc and exhibits increased binding affinity for human FcRn over an antibody having wild-type IgG Fc, by at least about 60 fold, preferably at least about 70 fold, more preferably at least about 80 fold, even more preferably at least about 100 fold, still more preferably at least about 125 fold, and most preferably at least about 150 fold to about 170 fold.

The N-glycosylation site in IgG is at Asn297 in the CH2 domain. Included for use in therapy herein are compositions of any eligible CD20 antibodies herein having an Fc region, wherein about 80-100% (and preferably about 90-99%) of the antibody in the composition comprises a mature core carbohydrate structure that lacks fucose, attached to the Fc region of the glycoprotein, or has reduced fucose content.

The expression “effective amount” with reference to a CD20 antibody (or other RA drug, such as methotrexate, MTX) refers to an amount of a medicament that is effective for treating RA. In particular, the effective amount of the CD20 antibody may increase the proportion of patients with ACR20 response at week 24, increase the proportion of patients with ACR50 response at week 24, increase the proportion of patients with ACR70 response at week 24, improve Disease Activity Score (DAS28-ESR) from baseline to week 24, improve EULAR response rates at week 24, improve ACR core set over time from baseline to week 48, improve SF-36 subscale and summary scores from baseline to week 48, improve FACIT fatigue assessment from baseline to week 48, increase proportion of patients achieving DAS28-ESR remission (DAS28-ESR<2.6) at week 24, increase proportion of patients achieving DAS28-ESR low disease activity (DAS28-ESR≦3.2) at week 24, increase proportion of patients with change from baseline in HAQ≧MCID (0.22) at week 24 and 48, and/or treat or prevent joint damage as compared to baseline prior to administration of such amount as determined, e.g., by radiographic or other testing.

As used herein, “rheumatoid arthritis” or “RA” refers to a recognized disease state that may be diagnosed according to the 2000 revised American Rheumatoid Association criteria for the classification of RA, or any similar criteria.

For the purposes herein, “tumor necrosis factor alpha” or “TNF-α” refers to a human TNF-α molecule comprising the amino acid sequence as described in Pennica et al., Nature, 312:721 (1984) or Aggarwal et al., JBC, 260:2345 (1985).

A “TNF inhibitor” herein is an agent that inhibits, to some extent, a biological function of TNF-α, generally through binding to TNF-α and neutralizing its activity. Examples of TNF-α inhibitors specifically contemplated herein are etanercept (ENBREL®), infliximab (REMICADE®), and adalimumab (HUMIRA™).

A “biologic-naïve” patient is one who has not previously been treated with a protein drug (particularly an antibody or immunoadhesin drug), such as a TNF-α inhibitor.

A “patient” herein is a human patient, eligible for treatment that is experiencing or has experienced one or more signs, symptoms, or other indicators of RA, whether, for example, newly diagnosed or previously diagnosed and now experiencing a non-response. In one embodiment the patient has “active” RA and may optionally be receiving background methotrexate.

The term “cytotoxic agent” as used herein refers to a substance that inhibits or prevents the function of cells and/or causes destruction of cells. The term is intended to include radioactive isotopes (e.g., At211, I131, I125, Y90, Re186, Re188, Sm153, Bi212, P32, and radioactive isotopes of Lu), chemotherapeutic agents, and toxins such as small-molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide, and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as MTX and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; XELODA® (capecitabine); ibandronate; CPT-11; topoisomerase inhibitor RFS 2000; difluoromethylornithine (DFMO); retinoic acid; esperamicins; capecitabine; and pharmaceutically acceptable salts, acids or derivatives of any of the above.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); NSAIDs; ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as MTX (oral or subcutaneous); anti-malarial agents such as chloroquine and hydroxychloroquine; sulfasalazine; leflunomide; cytokine antagonists such as cytokine antibodies or cytokine receptor antibodies including anti-interferon-α, -β, or -γ antibodies, anti-TNF-α antibodies (infliximab (REMICADE®) or adalimumab), anti-TNF-α immunoadhesin (etanercept), anti-TNF-β antibodies, anti-interleukin-2 (IL-2) antibodies and anti-IL-2 receptor antibodies, and anti-IL-6 receptor antibodies and antagonists (such as ACTEMRA™ (tocilizumab)); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 1990/08187); streptokinase; transforming growth factor-β (TGF-β); streptodornase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as anti-BAFF antibodies and anti-BR3 antibodies and zTNF4 antagonists (for review, see Mackay and Mackay, Trends Immunol., 23:113-5 (2002)); biologic agents that interfere with T cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some immunosuppressive agents herein are also DMARDs, such as MTX. Examples of preferred immunosuppressive agents herein include cyclophosphamide, chlorambucil, azathioprine, leflunomide, MMF, or MTX.

The term “cytokine” is a generic term for proteins released by one cell population that act on another cell as intercellular mediators. Examples of such cytokines are lymphokines, monokines; interleukins (ILs) such as IL-1, IL-1α, IL-1b, IL-2, IL-3, IL-4, IL-5, IL-6, IL-7, IL-8, IL-9, IL-11, IL-12, IL-15, including PROLEUKIN® rIL-2; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof. A “cytokine antagonist” is a molecule that inhibits or antagonizes such cytokines by any mechanism, including, for example, antibodies to the cytokine, antibodies to the cytokine receptor, and immunoadhesins.

The term “integrin” refers to a receptor protein that allows cells both to bind and respond to the extracellular matrix and is involved in a variety of cellular functions such as wound healing, cell differentiation, homing of tumor cells and apoptosis. They are part of a large family of cell adhesion receptors that are involved in cell-extracellular matrix and cell-cell interactions. Functional integrins consist of two transmembrane glycoprotein subunits, called α and β, which are non-covalently bound. The α subunits all share some homology to each other, as do the β subunits. The receptors always contain one a chain and one β chain. Examples include α6β1, α3β1, α7β1, the α4 chain such as α4β1, the β7 chain such as the β7 integrin subunit of α4β7 and/or αEβ7, LFA-1 etc. As used herein, the term “integrin” includes proteins from natural sources or from recombinant cell culture and biologically active equivalents of the native-sequence integrin, including synthetically produced small-molecule entities and pharmaceutically acceptable derivatives and salts thereof.

An “integrin antagonist” is a molecule that inhibits or antagonizes such integrins by any mechanism, including, for example, antibodies to the integrin. Examples of “integrin antagonists or antibodies” herein include an LFA-1 antibody, such as efalizumab (RAPTIVA®) commercially available from Genentech, or other CD11/11a and CD18 antibodies, or an α 4 integrin antibody such as natalizumab (ANTEGREN®) available from Biogen-IDEC, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US 2002/0042368), antibodies to αvβ3 integrin (EP 633945), anti-β7 antibodies such as rhuMAb β7 (US 2006/0093601) and MLN-02 (Millennium Pharmaceuticals), anti-α4 antibodies such as TYSABRI® (Biogen-IDEC-Élan), T0047 (GSK/Tanabe), CDP-323 (oral) (UCB), aza-bridged bicyclic amino acid derivatives (WO 2002/02556), etc.

Examples of “disease-modifying anti-rheumatic drugs” or “DMARDs” include hydroxycloroquine, sulfasalazine, MTX, leflunomide, etanercept, infliximab (plus oral and subcutaneous MTX), azathioprine, D-penicillamine, gold salts (oral), gold salts (intramuscular), minocycline, cyclosporine including cyclosporine A and topical cyclosporine, staphylococcal protein A (Goodyear and Silverman, J. Exp. Med., 197(9): 1125-39 (2003)), including salts and derivatives thereof, etc. A preferred DMARD herein is MTX.

Examples of “non-steroidal anti-inflammatory drugs” or “NSAIDs” include aspirin, acetylsalicylic acid, ibuprofen, naproxen, indomethacin, sulindac, tolmetin, COX-2 inhibitors such as celecoxib (CELEBREX®; 4-(5-(4-methylphenyl)-3-(trifluoromethyl)-1H-pyrazol-1-yl) benzenesulfonamide and valdecoxib (BEXTRA®), and meloxicam (MOBIC®), including salts and derivatives thereof, etc. Preferably, they are aspirin, naproxen, ibuprofen, indomethacin, or tolmetin.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as SOLU-MEDROL® methylprednisolone sodium succinate), dexamethasone or dexamethasone triamcinolone, hydrocortisone, and betamethasone. The preferred corticosteroids herein are prednisone, methylprednisolone, hydrocortisone, or dexamethasone.

The term “pharmaceutical formulation” refers to a sterile preparation that is in such form as to permit the biological activity of the medicament to be effective, and which contains no additional components that are unacceptably toxic to a subject to which the formulation would be administered.

A “sterile” formulation is aseptic or free from all living microorganisms and their spores.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

A “target audience” is a group of people or an institution to whom or to which a particular medicament is being promoted or intended to be promoted, as by marketing or advertising, especially for particular uses, treatments, or indications, such as individual patients, patient populations, readers of newspapers, medical literature, and magazines, television or internet viewers, radio or internet listeners, physicians, drug companies, etc.

### II. Modes for Carrying Out the Invention

The present invention provides, in at least one aspect, a method of treating a human rheumatoid arthritis (RA) patient comprising administering to the patient: (a) a CD20 antibody in an amount effective to treat the RA, and (b) at least one vaccine in an amount effective to mount an immune response to the protein vaccine. Preferably, the vaccine is a protein vaccine, and most preferably a tetanus toxoid vaccine.

Generally, the vaccine is administered to the patient after or following administration of the CD20 antibody. Optionally, the patient is B-cell depleted at the time of administration of the vaccine. For example, the vaccine may be administered from about 1 month to about one year after administration of the CD20 antibody. Most preferably, the vaccine is administered about six months after administration of the CD20 antibody. Each of the “administrations” here refers to any one or more doses of the CD20 antibody, and any one or more doses of the vaccine being administered from about one to twelve months or about six months apart.

Where the vaccine comprises a recall or memory antigen (e.g. tetanus toxoid vaccine), the immune response may constitute a 4-fold (or 2-fold) increase in anti-protein (e.g. anti-tetanus toxoid) titer following vaccination with the protein vaccine. The increase in titer may be measured or quantified as described in the examples herein, for instance about four weeks after vaccination.

Alternatively, or additionally, the invention concerns eliciting a delayed-type hypersensitivity (DTH) response in a RA patient treated with the CD20 antibody. Preferably, an antigen which results in a T-cell mediated response (such as C. Albicans or other skin test) is administered to the patient to generate the DTH response. Preferably, such antigen/DTH response is administered/elicted about six months after the CD20 antibody is administered.

Alternatively, or additionally, the invention concerns administering a polysaccharide vaccine (e.g. pneumococcal polysaccharide vaccine) and/or or neoantigen vaccine (e.g. Keyhole Limpet Hemocyanin, KLH) to the RA patient treated with the CD20 antibody in an amount effective to mount an immune response to the vaccine. Where the vaccine is a neoantigen vaccine, it is preferably administered in an amount effective to mount a primary humoral immune response to the neoantigen vaccine. The polysaccharide and/or neoantigen vaccine is/are preferably administered within about one year of the CD20 administration(s), for instance at about week 28 or about weeks 32, or 33.

In a preferred embodiment of the invention, the CD20 antibody is administered with one or more other drugs effective to treat RA. Most preferably, metotrexate (MTX) is combined with the CD20 antibody. According to this embodiment, the immune response (e.g. memory response) to the vaccine is about the same as that mounted in a patient treated with methotrexate only (i.e. MTX without a CD20 antibody). Optionally, the patient is further treated with one or more third, fourth, etc drugs, including one or more steroids or other immunosuppressive agents, such as methylprednisolone.

The CD20 antibody used in the therapeutic methods herein may be a chimeric, humanized, or human CD20 antibody. Examples include: rituximab, humanized 2H7, ofatumumab, veltuzumab, TRU-015, AME-133v, and GA101.

The invention also provides a method of mounting a 4-fold increase in anti-protein titer in a human rheumatoid arthritis (RA) patient following vaccination with a protein vaccine, comprising administering the protein vaccine to a RA patient who has been treated with a CD20 antibody, and measuring or quantifying the 4-fold increase in anti-protein titer.

Additionally, the invention provides a method of treating human patients with rheumatoid arthritis (RA) comprising treating a first group of the RA patients with a CD20 antibody, methotrexate, and a protein vaccine, and treating a second group of the RA patients with methotrexate and the vaccine but not the CD20 antibody, and determining that memory immune responses raised by the first and second groups of patients are about the same. Preferably the vaccine is tetanus toxoid and the immune response is 2-fold or 4-fold increase in anti-tetanus titer following vaccination with the tetanus toxoid vaccine. Desirably the first group of patients includes at least 50 patients.

These CD20 antibodies with which the patient or subject may be treated are produced using any suitable method, including those described below and in the examples herein.

The CD20 antibodies herein may be administered in any dose, provided it is effective to treat the patient. A physician having ordinary skill in the art can readily determine and prescribe the effective amount of the pharmaceutical composition required, depending on such factors as the particular CD20 antibody employed, prior clinical experience published in the literature on the CD20 antibody employed, the patient's characteristics and clinical history, the type and severity of RA, other medicines being given, and any side effects predicted. For example, the physician could start with doses of a CD20 antibody, employed in the pharmaceutical composition at levels lower than that required in order to achieve the desired therapeutic effect and gradually increase the dosage until the desired effect is achieved. The effectiveness of a given dose or treatment regimen of the CD20 antibody can be determined, for example, by assessing signs and symptoms and/or assessing inhibition of structural damage or of radiographic progression in the patient using the standard RA measures of efficacy.

The dose may be by weight or a fixed dose, preferably a fixed dose regardless of weight. An example of a weighted dose is 375 mg/m² weekly×4. As a general proposition, the effective amount of the antibody administered parenterally per dose will be in the range of about 20 mg to about 5000 mg, by one or more dosages, which can be translated to a dose by weight. Preferably the total dose is between about 50 and 4000 mg, preferably about 75 and 3000 mg, more preferably about 100 and 2000 mg, more preferably about 100 and 1000 mg, more preferably about 150 and 1000 mg, more preferably about 200 and 1000 mg, including doses of about 200, 300, 400, 500, 600, 700, 800, 900, 1000 mg, and 2000 mg. These doses may be given as a single dose or as multiple doses, for example, two to four doses. Such doses may be done by infusions, for example. More preferably, a CD20 antibody herein is administered at a dose of between about 200 and 1000 mg as a single dose or as two doses (preferably the doses are infusions). In a more preferred embodiment, the CD20 antibody is administered at about 200 mg×1 or 2, 300 mg×1 or 2, 400 mg×1 or 2, 500 mg×1 or 2, 600 mg×1 or 2, 700 mg×1 or 2, 800 mg×1 or 2, 900 mg×1 or 2, or 1000 mg×1 or 2. If administered in two doses, the drug in one embodiment is given on days 1 and 15, preferably intravenously, at the start of treatment.

Preferably, the frequency of dosings, if given in a multidose form, is about two to four doses within a period of about one month, or about two to three doses administered within a period of about 2 to 3 weeks.

As noted above, however, these suggested amounts of antibody are subject to a great deal of therapeutic discretion. The key factor in selecting an appropriate dose and scheduling is the result obtained, as indicated above. For example, relatively higher doses may be needed initially for the treatment of ongoing and acute RA or joint damage. To obtain the most efficacious results, the antibody is administered as close to the first sign, diagnosis, appearance, or occurrence of the RA as possible or during remissions of the RA.

In all the inventive methods set forth herein, the CD20 antibody may be unconjugated, such as a naked antibody, or may be conjugated with another molecule for further effectiveness, such as, for example, to improve half-life.

In another embodiment of all the methods herein, the CD20 antibody herein is the only medicament administered to the subject to treat the RA.

In an alternative aspect, one may administer a second medicament, as noted above, with the antibodies herein. The combined administration includes co-administration, using separate formulations or a single pharmaceutical formulation, and consecutive administration in either order, wherein preferably there is a time period while both (or all) active agents simultaneously exert their biological activities.

The second medicament includes, for example, an immunosuppressive agent, an antibody against CD20 other than the first medicament (that is the CD20 antibody being the first medicament), cytokine antagonist such as a cytokine antagonist, integrin antagonist (e.g., antibody), corticosteroid, or any combination thereof. The type of such second medicament depends on various factors, including the type of RA, the severity of the RA, the condition and age of the subject, the type and dose of first medicament employed, etc.

Examples of such additional medicaments include an immunosuppressive agent (such as mitoxantrone (NOVANTRONE®), methotrexate (MTX), cyclophosphamide, chlorambucil, leflunomide, and azathioprine), intravenous immunoglobulin (gamma globulin), lymphocyte-depleting therapy (e.g., mitoxantrone, cyclophosphamide, CAMPATH™ antibodies, anti-CD4, cladribine, rituximab, a 2H7 antibody, a polypeptide construct with at least two domains comprising a de-immunized, autoreactive antigen or its fragment that is specifically recognized by the Ig receptors of autoreactive B-cells (WO 2003/68822), total body irradiation, bone marrow transplantation), integrin antagonist or antibody (e.g., an LFA-1 antibody such as efalizumab/RAPTIVA® commercially available from Genentech, or an α 4 integrin antibody such as natalizumab/ANTEGREN® available from Biogen, or others as noted above), drugs that treat symptoms secondary or related to RA and/or joint damage such as those noted herein, steroids such as corticosteroid (e.g., prednisolone, methylprednisolone such as SOLU-MEDROL™ methylprednisolone sodium succinate for injection, prednisone such as low-dose prednisone, dexamethasone, or glucocorticoid, e.g., via joint injection, including systemic corticosteroid therapy), non-lymphocyte-depleting immunosuppressive therapy (e.g., MMF or cyclosporine), a TNF-α inhibitor such as an antibody to TNF-α, DMARD, NSAID, plasmapheresis or plasma exchange, trimethoprim-sulfamethoxazole (BACTRIM™, SEPTRA™), mycophenolate mofetil, H2-blockers or proton-pump inhibitors (during the use of potentially ulcerogenic immunosuppressive therapy), levothyroxine, cyclosporin A (e.g. SANDIMMUNE®), somatostatin analogue, a DMARD or NSAID, cytokine antagonist such as antibody, anti-metabolite, immunosuppressive agent, rehabilitative surgery, radioiodine, thyroidectomy, anti-IL-6 receptor antagonist/antibody (e.g., ACTEMRA™ (tocilizumab)), or another B-cell antagonist such as BR3-Fc, TACI-Ig, anti-BR3 antibody, anti-CD40 receptor or anti-CD40 ligand (CD154), agent blocking CD40-CD40 ligand, epratuzumab (anti-CD22 antibody), lumiliximab (anti-CD23 antibody), or an antibody directed against human CD20 other than rituximab or the CD20 antibodies used herein, such as a 2H7 antibody.

Preferred such medicaments include gamma globulin, an integrin antagonist, anti-CD4, cladribine, trimethoprimsulfamethoxazole, an H2-blocker, a proton-pump inhibitor, cyclosporine, a TNF-α inhibitor, a DMARD, an NSAID (to treat, for example, musculoskeletal symptoms), levothyroxine, a cytokine antagonist (including cytokine-receptor antagonist), an anti-metabolite, an immunosuppressive agent such as MTX or a corticosteroid, a bisphosphonate, and another antagonist to a B-cell surface marker, such as, for example, a small molecule to CD20, a CD22 antibody, a BR3 antibody, lumiliximab (anti-CD23 antibody), BR3-Fc, or TACI-Ig.

The more preferred such medicaments are an immunosuppressive agent such as MTX or a corticosteroid, a DMARD, a different antibody against CD20 than the first medicament, an integrin antagonist, a NSAID, a cytokine antagonist, a bisphosphonate, or a combination thereof.

In one particularly preferred embodiment, the second medicament is a DMARD, which is preferably selected from the group consisting of auranofin, chloroquine, D-penicillamine, injectable gold, oral gold, hydroxychloroquine, sulfasalazine, myocrisin, and MTX.

In another such embodiment, the second medicament is a NSAID, which is preferably selected from the group consisting of: fenbufen, naprosyn, diclofenac, etodolac and indomethacin, aspirin and ibuprofen.

In a further such embodiment, the second medicament is an immunosuppressive agent, which is preferably selected from the group consisting of etanercept, infliximab, adalimumab, leflunomide, anakinra, azathioprine, MTX, and cyclophosphamide.

In other preferred embodiments, the second medicament is selected from the group consisting of anti-α4, etanercept, infliximab, etanercept, adalimumab, kinaret, efalizumab, OPG, RANK-Fc, anti-RANKL, pamidronate, alendronate, actonel, zolendronate, rituximab, a 2H7 antibody, clodronate, MTX, azulfidine, hydroxychloroquine, doxycycline, leflunomide, SSZ, prednisolone, interleukin-1 receptor antagonist, prednisone, and methylprednisolone.

In still preferred embodiments, the second medicament is selected from the group consisting of MTX, infliximab, a combination of infliximab with MTX, etanercept, a corticosteroid, cyclosporin A, azathioprine, auranofin, hydroxychloroquine (HCQ), a combination of prednisolone with MTX and SSZ, a combination of MTX with SSZ and HCQ, a combination of cyclophosphamide with azathioprine and HCQ, and a combination of adalimumab with MTX. If the second medicament is a corticosteroid, preferably it is prednisone, prednisolone, methylprednisolone, hydrocortisone, or dexamethasone. Also, preferably, the corticosteroid is administered in lower amounts than are used if the CD20 antibody is not administered to a subject treated with a corticosteroid. Most preferably, the second medicament is MTX.

All these second medicaments may be used in combination with each other or by themselves with the first medicament, so that the expression “second medicament” as used herein does not mean it is the only medicament besides the first medicament, respectively. Thus, the second medicament need not be one medicament, but may constitute or comprise more than one such drug.

These second medicaments as set forth herein are generally used in the same dosages and with administration routes as used hereinbefore or about from 1 to 99% of the heretofore-employed dosages. If such second medicaments are used at all, preferably, they are used in lower amounts than if the first medicament were not present, especially in subsequent dosings beyond the initial dosing with the first medicament, so as to eliminate or reduce side effects caused thereby.

The present application contemplates re-treatments with the CD20 antibody. For such re-treatment methods, where a second medicament is administered in an effective amount with an antibody exposure, it may be administered with any exposure, for example, only with one exposure, or with more than one exposure. In one embodiment, the second medicament is administered with the initial exposure. In another embodiment, the second medicament is administered with the initial and second exposures. In a still further embodiment, the second medicament is administered with all exposures. It is preferred that after the initial exposure, such as of steroid, the amount of such second medicament is reduced or eliminated so as to reduce the exposure of the subject to an agent with side effects such as prednisone, prednisolone, methylprednisolone, and cyclophosphamide.

The combined administration of a second medicament includes co-administration (concurrent administration), using separate formulations or a single pharmaceutical formulation, and consecutive administration in either order, wherein preferably there is a time period while both (or all) active agents (medicaments) simultaneously exert their biological activities.

The CD20 antibody herein is administered by any suitable means, including parenteral, topical, intraperitoneal, intrapulmonary, intranasal, and/or intralesional administration. Parenteral infusions include intramuscular, intravenous (i.v.), intraarterial, intraperitoneal, or subcutaneous (s.c.) administration. In addition, the CD20 antibody may suitably be administered by pulse infusion, e.g., with declining doses of the CD20 antibody. Preferably, the dosing is given by i.v. or s.c. administration. Whether the administration is i.v. or s.c. will depend on many factors, including the type of CD20 antibody employed, the clinical history of the patient, the particular dosing and scheduling, etc. In some cases it may be preferable to give the antibody by s.c. rather than i.v. administration.

If multiple exposures of the CD20 antibody are provided, each exposure may be provided using the same or a different administration means. In one embodiment, each exposure is by i.v. administration. In another embodiment, each exposure is given by s.c. administration. In yet another embodiment, the exposures are given by both i.v. and s.c. administration.

In one embodiment, the CD20 antibody is administered as a slow i.v. infusion rather than an i.v. push or bolus. For example, a steroid such as prednisolone or methylprednisolone (e.g., about 80-120 mg i.v., more specifically about 100 mg i.v.) is administered about 30 minutes prior to any infusion of the CD20 antibody. The CD20 antibody is, for example, infused through a dedicated line.

For the initial dose of a multi-dose exposure to the CD20 antibody, or for the single dose if the exposure involves only one dose, such infusion is preferably commenced at a rate of about 50 mg/hour. This may be escalated, e.g., at a rate of about 50 mg/hour increments every about 30 minutes to a maximum of about 400 mg/hour. However, if the subject is experiencing an infusion-related reaction, the infusion rate is preferably reduced, e.g., to half the current rate, e.g., from 100 mg/hour to 50 mg/hour. Preferably, the infusion of such dose of CD20 antibody (e.g., an about 1000-mg total dose) is completed at about 255 minutes (4 hours 15 min.). Optionally, the subjects receive a prophylactic treatment of acetaminophen/paracetamol (e.g., about 1 g) and diphenhydramine HCl (e.g., about 50 mg or equivalent dose of similar agent) by mouth about 30 to 60 minutes prior to the start of an infusion.

If more than one infusion (dose) of CD20 antibody is given to achieve the total exposure, the second or subsequent CD20 antibody infusions in this infusion embodiment are preferably commenced at a higher rate than the initial infusion, e.g., at about 100 mg/hour. This rate may be escalated, e.g., at a rate of about 100 mg/hour increments every about 30 minutes to a maximum of about 400 mg/hour. Subjects who experience an infusion-related reaction preferably have the infusion rate reduced to half that rate, e.g., from 100 mg/hour to 50 mg/hour. Preferably, the infusion of such second or subsequent dose of CD20 antibody (e.g., an about 1000-mg total dose) is completed by about 195 minutes (3 hours 15 minutes).

Once the patient population most responsive to treatment with the CD20 antibody has been identified, treatment with the antibody herein, alone or in combination with other medicaments, results in an improvement in the RA, including signs or symptoms thereof. For instance, such treatment may result in an improvement in ACR measurements relative to a patient treated with the second medicament only (e.g., an immunosuppressive agent such as MTX), and/or may result in an objective response (partial or complete, preferably complete) as measured by ACR. Moreover, treatment with the combination of an antibody herein and at least one second medicament(s) preferably results in an additive, more preferably synergistic (or greater than additive) therapeutic benefit to the patient. Preferably, in this combination method the timing between at least one administration of the second medicament and at least one administration of the antibody herein is about one month or less, more preferably, about two weeks or less.

For purposes of the methods herein, success of treatment is determined as set forth above. Clinical improvement is preferably determined by assessing the number of tender or swollen joints, conducting a global clinical assessment of the patient, assessing erythrocyte sedimentation rate, assessing the amount of C-reactive protein level, or using composite measures of disease activity (disease response) such as the DAS-28, ACR-20, -50, or -70 scores.

In a further embodiment, the subject does not have a malignancy, including a B-cell malignancy, solid tumors, hematologic malignancies, or carcinoma in situ (except basal cell and squamous cell carcinoma of the skin that have been excised and cured). Additionally, the patient preferably does not have another autoimmune disease other than RA.

### III. Production of CD20 Antibodies

The preferred CD20 antibodies herein are generally manufactured as follows.

Rituximab (RITUXAN®)

Rituximab is a chimeric CD20 therapeutic antibody that first received FDA approval in November 1997 for the treatment of relapsed or refractory, low-grade or follicular, CD20-positive, B-cell non-Hodgkin's lymphoma (NHL). It was also approved in the European Union under the trade name MabThera® in June 1998. In February 2006, Rituxan also received FDA approval in combination with MTX to reduce signs and symptoms in adult patients with moderately-to-severely-active RA who have had an inadequate response to one or more TNF antagonist therapies. Rituxan is the first treatment for RA that selectively targets immune cells known as CD20-positive B-cells. Rituxan does not target the entire immune system. The structure of rituximab antibody (also designated C2B8) and exemplary methods for its production via recombinant expression in Chinese Hamster Ovary (CHO) cells are disclosed in U.S. Pat. No. 5,736,137 (Anderson et al.). The product is also commercially available from Genentech and Roche.

Rituximab displays antibody-dependent cellular cytotoxicity (ADCC) in vitro. Potent complement-dependent cytotoxicity (CDC) activity has also been observed for rituximab on lymphoma cells and cell lines and in certain mouse xenograft models. Several CD20 antibodies, including rituximab, have also been shown to induce apoptosis in vitro when crosslinked by a secondary antibody or by other means.

Other chimeric, humanized, or human antibodies with biological activities also displayed by rituximab can be used herein and are described below.

### Humanized 2H7

Sequences of various humanized 2H7 antibodies have been described above. Further information regarding humanized 2H7 antibody structures, and exemplary methods for production of such antibodies can be found in: US2006/0034835, US2006/0024300, US 2006/0067930, and US 2006/0246004.

### Ofatumumab (HUMAX-CD20™)

Ofatumumab (2F2) may be prepared, for example, in accordance with the procedures described in US 2004/0167319, the disclosure of which is specifically incorporated herein by reference. The amino acid sequences of the second heavy-chain variable region and the light-chain variable region are also depicted in FIG. 53 of US 2004/0167319 with their designated CDR regions.

Examples 1-3 of US 2004/0167319 disclose the specifics of preparation of 2F2. Specifically, fully human monoclonal antibodies to CD20 were prepared using HCo7 and KM mice that express human antibody genes.

One of the hybridoma cell lines generated expressed 2F2, a human monoclonal IgG1, κ antibody with the nucleotide sequences SEQ ID NOS:1 and 3 and the amino acid sequences SEQ ID NOS:2 and 4 of US 2004/0167319.

IMMU-106 (hA20 or Veltuzumab)

FIG. 5 of US 2003/0219433 discloses the nucleotide sequences of hA20 light chain V genes, (hA20Vk) (FIG. 5A), and heavy chain V genes, hA20VH1 (FIG. 5B) and hA20VH2 (FIG. 5C), as well as the adjacent flanking sequences of the VKpBR2 (FIG. 5A) and VHpBS2 (FIGS. 5B and 5C) staging vectors, respectively. The non-translated nucleotide sequences are shown in lower-case letters. The restriction sites used for subcloning are underlined and indicated. The secretion signal peptide sequence is indicated by a double underline. Amino acid sequences are given as single-letter codes below the corresponding nucleotide sequence. The Kabat numbering scheme was used for amino acid residues. Amino acid residues numbered by a letter represent the insertion residue according to Kabat, and have the same number as that of the previous residue.

Methods for constructing veltuzumab are described, for example, in US 2003/0219433, the disclosure of which is specifically incorporated herein by reference.

### Immunopharmaceutical (TRU-015)

CD20-specific SMIPs are described generally in US 2003/133939, US 2003/0118592, and US 2005/0136049, the disclosures of which are specifically incorporated herein by reference. Production of an exemplary CD20-specific SMIP, TRU-015, is described, for example, in US 2007/0059306, the disclosure of which is specifically incorporated herein by reference, and below.

TRU-015 is a recombinant (murine/human) single-chain protein that binds to the CD20 antigen. The binding domain was based on a publicly available human CD20 antibody sequence. The binding domain is connected to the effector domain, the CH2 and CH3 domains of human IgG1, through a modified CSS hinge region. TRU-015 exists as a dimer in solution and the dimer has a theoretical molecular weight of approximately 106,000 daltons.

TRU-015 may be cultured in a bioreactor using appropriate media and then purified using a series of chromatography and filtration steps, including, for example, a step employing a virus reduction filter. The material may then be concentrated and formulated with suitable excipients such as, for example, sodium phosphate (e.g., 20 mM) and sucrose (e.g., 240 mM) at an appropriate physiologically acceptable pH, for example, pH 6-7, more preferably 6.0. The composition may then be filtered before filling into vials, such as glass vials, at a concentration, for example, of 10 mg/mL. Each glass vial may contain, for example, 5 mL of TRU-015 (50 mg/vial).

### AME CD20 Antibodies

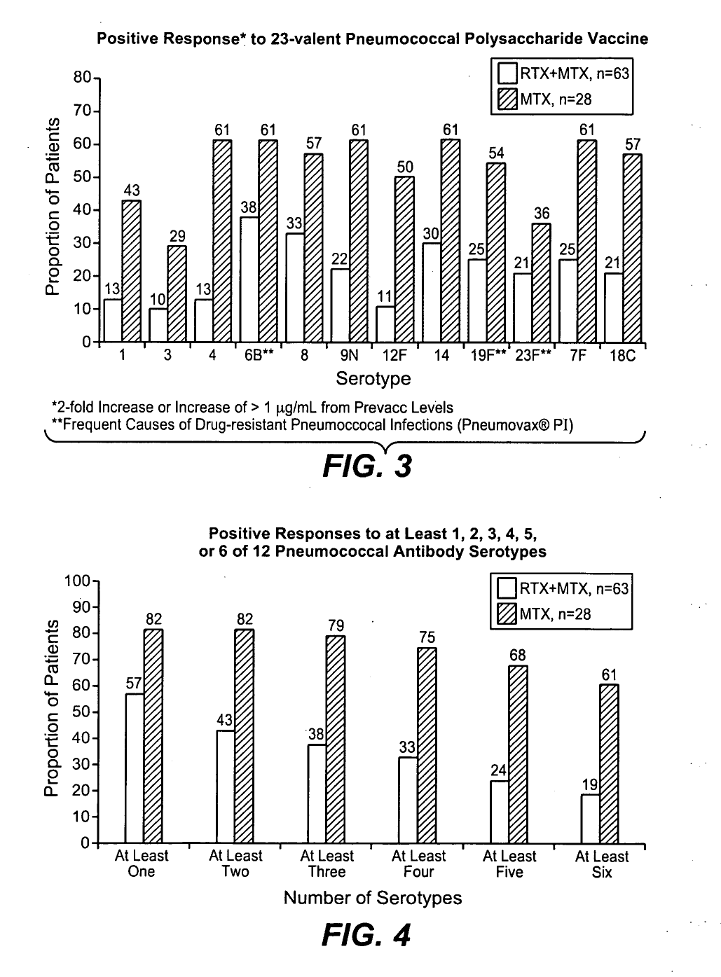

The CD20-binding antibody AME 33 is prepared as described, for example, in US 2005/0025764 and US 2006/0251652, the disclosures of which are specifically incorporated herein by reference. The polynucleotide and amino acid sequences for the heavy- and light-chain variable regions of AME 33 are presented in both these applications as FIGS. 2-3 (SEQ ID NOS:59-62). The amino acid sequences for the light- and heavy-chain variable regions of AME 33 are respectively set forth above as SEQ ID NOS:13 and 15.

Example 1 of US 2005/0025764 describes the preparation of AME 33 in detail, including setting forth the CDR regions for each variable domain. The light- and heavy-chain variable regions for the CD20-binding molecule AME 33 may be combined with light- and heavy-chain constant regions and expressed as Fabs or full antibodies (e.g., IgG). For example, FIGS. 10 and 11 of US 2005/0025764 show the complete light and heavy chains for AME 33, which include the light- and heavy-chain constant regions, which are underlined in FIGS. 10A and 11A. Alternatively, AME 33 may contain the heavy-chain constant regions shown in those two figures except with an amino acid substitution in the Fc region. In particular, the heavy-chain constant region shown in FIG. 11 of that patent application may contain a D280H mutation or a K290S mutation (FIG. 11A shows positions 280 and 290 in bold, without the mutations). FIG. 11B shows a bold and underlined “GAC.”

The CD20-binding antibody AME 133 is prepared as disclosed, for example, in US 2005/0136044, the disclosure of which is specifically incorporated herein by reference, including Example VII. The polynucleotide and amino acid sequences for the light-chain variable region of AME 133 are set forth as SEQ ID NOS:197 and 198, respectively, in US 2005/0136044.

The polypeptide representing AME 133v, a fusion protein prepared from the AME 133 Fab region fused to modified BChE variant L530, is also disclosed in US 2005/0136044, see, e.g. SEQ ID NO:19 of US 2005/0136044.

Humanized Type II CD20 IgG1 Antibody with Glycoengineered Fc Region (GA101)

The molecule GA101 is a humanized type II CD20 IgG1 antibody. It is humanized by grafting CDR sequences from the murine monoclonal antibody B-ly1 onto framework regions with fully human IgG1-kappa germline sequences. Also, the Fc region-carbohydrates of this antibody are glycoengineered using GLYCOMAB™ technology described in WO 2004/065540 (the disclosure of which is specifically incorporated herein by reference), leading to bisected afucosylated Fc region-carbohydrates. GA101 is BHH2-KV1-GE, the preparation of which is described, for example, in US 2005/0123546, the disclosure of which is specifically incorporated herein by reference. See especially Example 2 thereof.

Important properties of the humanized B-Ly1 antibody are that it is a type II CD20 antibody as defined in Cragg and Glennie, Blood, 103(7):2738-2743 (2004). It therefore did not induce, upon binding to CD20, any significant resistance to non-ionic detergent extraction of CD20 from the surface of CD20+human cells, using the assay described for this purposes in Polyak and Deans, Blood, 99(9):3256-3262 (2002). According to US 2005/0123546, the humanized B-Ly1 antibody induced less resistance to non-ionic detergent extraction of CD20 than the C2B8 antibody (another CD20 antibody with identical sequence to rituximab (see US 2003/0003097, Reff). As expected of a type II CD20 antibody, the humanized B-Ly1 did not have any significant complement-mediated lysis activity. The humanized B-Ly1 antibody was very potent in the homotypic aggregation assay. In this assay CD20-positive human cells, Daudi cells, were incubated in cell culture medium for up to 24 hours at 37° C. in a 5% CO₂ atmosphere in a mammalian cell incubator, with the antibody at a concentration of 1 microgram per ml and in parallel at a concentration of 5 micrograms per ml. The aggregates were reported to be larger that those induced by addition of the C2B8 control antibody. In addition, and consistent with the antibody being CD20 type II, the humanized B-Ly1 antibody was reported to induce higher levels of apoptosis when CD20-positive human cells were incubated therewith, relative to a control under identical conditions using the C2B8 chimeric IgG1 antibody.

Glycoengineered variants of the humanized antibodies were produced by co-expression of GnTIII glycosyltransferase, together with the antibody genes, in mammalian cells. This led to an increase in the fraction of non-fucosylated oligosaccharides attached to the Fc region of the antibodies, including bisected non-fucosylated oligosaccharides, as has been described in WO 2004/065540 (FIGS. 17-19). The glycoengineered antibodies had significantly higher levels of binding to human FcγRIII receptors and ADCC activity as well, relative to the non-glycoengineered antibody and relative to the C2B8 antibody. The humanized B-Ly1 antibody was also more potent at inducing human B-cell depletion in a whole blood assay than the control C2B8 antibody. This was true both for the non-glycoengineered B-Ly1 antibody and for the glycoengineered version of it. The glycoengineered antibody was approximately 1000-fold more potent than the C2B8 control CD20 antibody in depleting B-cells in the whole blood assay.

### IV. Pharmaceutical Formulations

Therapeutic formulations of the antibodies used in accordance with the present invention are prepared for storage by mixing the antibody having the desired degree of purity with optional pharmaceutically acceptable carriers, excipients, or stabilizers in the form of lyophilized formulations or aqueous solutions. For general information concerning formulations, see, e.g., Gilman et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Eastori, Pa.; Avis et al., (eds.) (1993) Pharmaceutical Dosage Forms: Parenteral Medications Dekker, New York; Lieberman et al., (eds.) (1990) Pharmaceutical Dosage Forms: Tablets Dekker, New York; and Lieberman et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, or polyethylene glycol (PEG).

Exemplary CD20 antibody formulations are described in the patent applications cited above that describe the antibodies herein, including those cited in the background section herein, the disclosures of all of which are specifically incorporated by reference herein.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystallized forms of the antibodies are also contemplated. See, for example, US 2002/0136719A1 (Shenoy et al.).

The formulation herein may also contain more than one active compound (a second medicament as defined above), preferably those with complementary activities that do not adversely affect each other. The type and effective amounts of such medicaments depend, for example, on the amount and type of CD20 antibody present in the formulation, and clinical parameters of the subjects. The preferred such second medicaments are noted herein.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed, for example, in Remington's Pharmaceutical Sciences 16th edition, Osol, A. Ed. (1980).

Examples of sustained-release preparations applicable herein include semi-permeable matrices of solid hydrophobic polymers containing the CD20 antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### V. Articles of Manufacture

Articles of manufacture containing materials useful for the treatment of the RA are provided herein. The article of manufacture comprises a container and a label or package insert on or associated with the container. In this aspect, the package insert is on or associated with the container. Suitable containers include, for example, bottles, vials, syringes, etc. The containers may be formed from a variety of materials such as glass or plastic. The container holds or contains the CD20 antibody that is effective for treating the RA and may have a sterile access port (for example, the container may be an i.v. solution bag or a vial having a stopper pierceable by a hypodermic injection needle). At least one active agent in the composition is the CD20 antibody. The label or package insert indicates that the composition is used for treating RA in a human patient eligible for treatment with specific guidance regarding dosing amounts and intervals of antibody and any other medicament being provided. The label or package insert further indicates that the patient so-treated can be further treated with a one or more vaccines including: a protein vaccine (e.g. tetanus toxoid vaccine) in an amount effective to mount a memory immune response to the vaccine; an antigen which results in a T-cell mediated response (e.g. C. Albicans) in an amount effective to elicit a delayed-type hypersensitivity (DTH) response in the patient; and/or a polysaccharide vaccine (e.g. pneumococcal polysaccharide vaccine) in an amount effective to mount a immune response to the polysaccharide vaccine; and/or a neoantigen vaccine (e.g. Keyhole Limpet Hemocyanin, KLH) in an amount effective to mount a primary humoral immune response to the neoantigen vaccine.

The article of manufacture may further comprise a second container comprising a pharmaceutically acceptable diluent buffer, such as bacteriostatic water for injection (BWFI), phosphate-buffered saline, Ringer's solution, and dextrose solution. The article of manufacture may further include other materials desirable from a commercial and user standpoint, including other buffers, diluents, filters, needles, and syringes.

The kits and articles of manufacture of the present invention also include information, for example in the form of a package insert or label, indicating that the composition is used for treating RA. The insert or label may take any form, such as paper or electronic media, for example, a magnetically recorded medium (e.g., floppy disk) or a CD-ROM. The label or insert may also include other information concerning the pharmaceutical compositions and dosage forms in the kit or article of manufacture.

Generally, such information aids patients and physicians in using the enclosed pharmaceutical compositions and dosage forms effectively and safely. For example, the following information regarding the antibody may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

In another aspect, the invention provides a method of providing a pharmaceutical composition for a human rheumatoid arthritis (RA) patient, comprising combining a container holding a pharmaceutically acceptable composition comprising a CD20 antibody with a package insert, wherein the package insert promotes the use of the composition to treat a RA patient who is able to mount an effective memory immune response to a protein vaccine administered following administration of the CD20 antibody.

### VI. Methods of Advertising

The invention herein also encompasses a method for advertising a CD20 antibody comprising promoting the use of the CD20 antibody for treating a human rheumatoid arthritis (RA) patient, wherein the RA patient is able to mount an effective memory immune response to a protein vaccine administered following administration of the CD20 antibody. Preferably the protein vaccine is a tetanus toxoid vaccine. In one embodiment, such effective memory immune response comprises a 2-fold rise in anti-protein (e.g. anti-tetanus) titer or a 4-fold rise in anti-protein (e.g. anti-tetanus) titer. Optionally, the CD20 antibody is promoted for treating a human RA patient who is able to mount an immune response to a polysaccharide vaccine (e.g. pneumococcal polysaccharide vaccine) and/or neoantigen vaccine (e.g. Keyhole Limpet Hemocyanin, KLH).

Advertising is generally paid communication through a non-personal medium in which the sponsor is identified and the message is controlled. Advertising for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The advertising and promotion of the treatment methods herein may be accomplished by any means. Examples of advertising media used to deliver these messages include television, radio, movies, magazines, newspapers, the internet, and billboards, including commercials, which are messages appearing in the broadcast media. Advertisements also include those on the seats of grocery carts, on the walls of an airport walkway, and on the sides of buses, or heard in telephone hold messages or in-store PA systems, or anywhere a visual or audible communication can be placed. More specific examples of promotion or advertising means include television, radio, movies, the internet such as webcasts and webinars, interactive computer networks intended to reach simultaneous users, fixed or electronic billboards and other public signs, posters, traditional or electronic literature such as magazines and newspapers, other media outlets, presentations or individual contacts by, e.g., e-mail, phone, instant message, postal, courier, mass, or carrier mail, in-person visits, etc.

The type of advertising used will depend on many factors, for example, on the nature of the target audience to be reached, e.g., hospitals, insurance companies, clinics, doctors, nurses, and patients, as well as cost considerations and the relevant jurisdictional laws and regulations governing advertising of medicaments and diagnostics. The advertising may be individualized or customized based on user characterizations defined by service interaction and/or other data such as user demographics and geographical location.

Further details of the invention are illustrated by the following non-limiting example. The disclosures of all citations in the specification are expressly incorporated herein by reference.

### EXAMPLE

### A Randomized, Open-Label, Study to Evaluate the Effects of Rituximab on Immune Response in Subjects with Active Rheumatoid Arthritis Receiving Background Methotrexate

This example provides the data for a Phase II, randomized, open-label, multicenter study designed to evaluate immune response to vaccines after administration of 1000 mg of rituximab on Days 3 and 17 in subjects with active RA who were receiving background MTX. Following screening, approximately 100 adult volunteers were randomized 2:1 (active:control) to one of two groups: Group A (active group; approximately 66 subjects) and Group B (control group; approximately 33 subjects). Subjects were also stratified by study site and age (18-50 years and 51-65 years). Subjects with active RA treated with rituximab in combination with MTX (Group A—Active group) were compared with subjects treated with MTX alone (Group B—Control group).

This study included a screening period, a 36-week treatment period, an optional extension retreatment, a safety follow-up (SFU) period, and a B-cell follow-up period. During the treatment period, Group A subjects continued their background MTX (10-25 mg/wk) and received 1000 mg of open-label rituximab on Days 3 and 17; methylprednisolone 100 mg intravenous (IV) was administered before each infusion of rituximab. Group A subjects completed the treatment period at Week 36. During the primary study period, Group B subjects continued background MTX (10-25 mg/wk) and did not receive any rituximab for the first 12 weeks of the study. At Week 12 (the end of the primary study period), Group B subjects, if eligible, could then choose to receive one course of rituximab (1000 mg IV×2, 14 days apart) for treatment of active RA. Group B subjects who did not qualify for and/or did not choose treatment with rituximab completed the study at Week 12.

Subjects in Groups A and B who completed the 36-week treatment period had the option for retreatment if they met the optional extension retreatment criteria, and chose to receive retreatment.

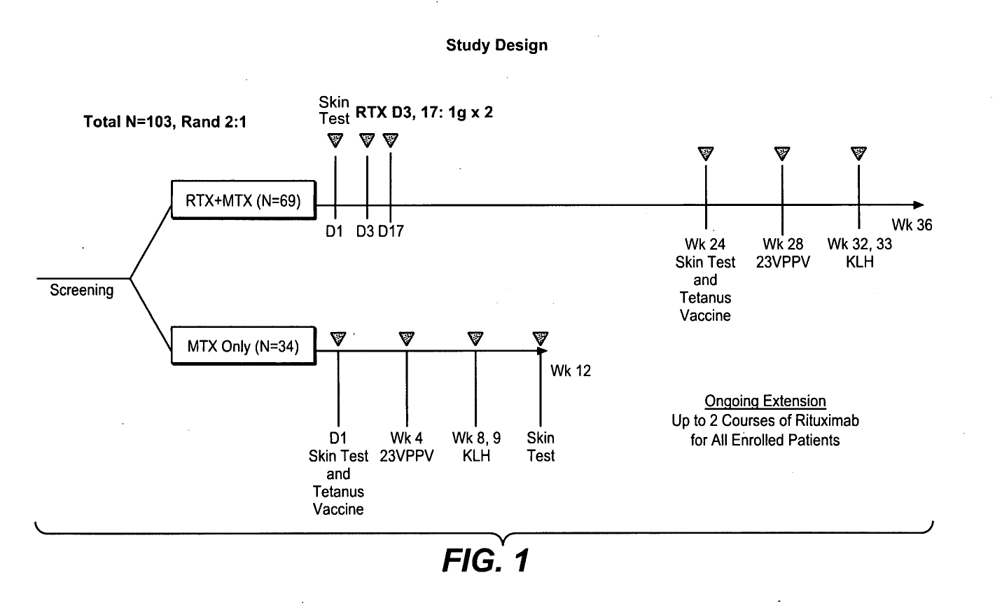

The Study Design is shown in FIG. 1.

### Vaccines Studied

FIG. 2 summarizes the specific antigens/vaccines tested. In particular, the following vaccines were studied:

### Tetanus Toxoid Adsorbed Vaccine

Tetanus toxoid adsorbed vaccine is indicated for the prevention of tetanus. In this study, tetanus toxoid adsorbed vaccine was used to assess whether rituximab affected antibody production to an antigen to which individuals had an existing immunity.

### The 23-Valent Pneumococcal Polysaccharide Vaccine

The 23-valent pneumococcal polysaccharide vaccine (PNEUMOVAX®) is indicated for vaccination against pneumococcal disease caused by those pneumococcal types included in the vaccine. It was chosen for this study to assess antibody production for a clinically relevant antigen that was unknown to most individuals. The 23-valent pneumococcal polysaccharide vaccine (PNEUMOVAX®) was administered in the deltoid muscle as a single intramuscular (IM) injection.

### Keyhole Limpet Hemocyanin

Keyhole Limpet Hemocyanin (KLH) is a high molecular weight respiratory metalloprotein found in the hemolymph of many mollusks and crustaceans. KLH is an investigational agent and is not approved for use as a vaccine; however, it has been used to evaluate immune response in clinical trials. In this study, it was used to test primary humoral response following B cell depletion with rituximab.

Candida albicans Skin Test

In this study, T-cell memory with rituximab treatment in RA was evaluated by eliciting a delayed hypersensitivity response by intradermal skin testing with the recall antigen Candida albicans.

### Assay Methods

### Rituximab Pharmacokinetic Assay

The rituximab pharmacokinetic ELISA measures rituximab levels in human serum samples. It uses affinity purified polyclonal goat anti-rituximab as a capturing reagent and goat antibody to mouse IgG F(ab)2 conjugated to horseradish peroxidase as a detection reagent.

### Rituximab HACA Assay

The rituximab human anti-chimeric antibody (HACA) ELISA is a bridging assay, which uses rituximab as the capturing reagent and biotinylated-rituximab and strepavidin-HRP for detection. The assay uses a calibrator curve prepared with affinity purified polyclonal goat antibodies to rituximab; therefore, results from this assay were reported relative to this polyclonal antibody in terms of relative units (RU).

### Tetanus Antibody Assay

The tetanus antibody test was used to measure anti-tetanus antibody levels in human serum samples. The tetanus antibody test is an ELISA that uses tetanus toxoid as a capturing reagent and alkaline phosphatase-conjugated anti-human IgG for detection. Results were reported in international units (IU)/mL.

### Pneumococcal Antibody Assay

The pneumococcal antibody assay was used to measure anti-pneumococcal antibody levels in human serum samples. The pneumococcal antibody assay is a fluoroimmunoassay that uses a LUMINEX MULTIPLEX™ platform. Purified capsular polysaccharides isolated from 12 serotypes of S. pneumoniae are covalently attached to microbeads and used as a capturing reagent. Phycoerythrin conjugated anti-human IgG was used for detection. Results were reported in microgram of IgG/mL.

### KLH Antibody Assay

A KLH antibody assay was used to measure anti-KLH antibody levels in human serum samples. The KLH antibody assay is an enzyme-linked immunosorbant assay (ELISA) format using KLH as the plate coat and anti-human IgG-horseradish peroxidase for detection. Results were reported in titer units.

### Outcome Measures

### Primary Outcome Measure

The primary outcome measure was the proportion of subjects in Groups A and B with a positive response to tetanus toxoid adsorbed vaccine measured 4 weeks after tetanus toxoid adsorbed vaccine administration.

The proportion of subjects in Group A with positive responses to tetanus toxoid adsorbed vaccine measured 4 weeks after the tetanus toxoid adsorbed vaccine were compared with the proportion of subjects in Group B with positive responses to tetanus toxoid adsorbed vaccine measured 4 weeks after the tetanus toxoid adsorbed vaccine.

For subjects with prevaccination tetanus antibody titers <0.1 IU/mL, a response to the booster immunization was defined as an antibody titer ≧0.2 IU/mL measured 4 weeks after the immunization. For subjects with prevaccination tetanus antibody titers ≧0.1 IU/mL, positive response to the booster immunization was defined as a 4-fold increase in antibody titer measured 4 weeks after the immunization.

Prevaccination levels were those obtained immediately prior to receipt of a vaccine.

In addition, as an exploratory analysis, a logistic regression model was used to investigate the interaction between treatment and factors such as age, sex, background corticosteroid use, and MTX dose.

### Secondary Outcome Measures

The secondary outcome measures were as follows: the proportion of subjects in Groups A and B with a 2-fold increase in tetanus antibody titers, or with tetanus antibody titers ≧0.2 IU/mL, measured 4 weeks after the immunization of subjects with prevaccination tetanus antibody titers ≧0.1 IU/mL or with prevaccination tetanus antibody titers <0.1 IU/mL, respectively; the proportion of subjects in Groups A and B with positive responses against an individual anti-pneumococcal antibody serotype measured 4 weeks after the 23-valent pneumococcal polysaccharide vaccine (12 serotypes); the proportion of subjects in Groups A and B with positive responses against at least 50% of the serotypes (≧6/12) measured 4 weeks after pneumococcal polysaccharide vaccine; levels of anti-tetanus antibody in subjects in Groups A and B measured immediately prior to and 4 weeks after a booster vaccine; levels of anti-pneumococcal antibody to 12 serotypes in subjects in Groups A and B measured immediately prior to and 4 weeks after vaccination; levels of anti-KLH antibody in subjects in Groups A and B measured immediately prior to the first administration of KLH and 4 weeks after the first administration of KLH; and the proportion of subjects who maintain a positive response to Candida albicans from Day 1 to 24 weeks (Group A) and Day 1 to 12 weeks (Group B), as measured by the diameter of induration.

The following secondary endpoints were assessed:

1. The proportion of subjects in Groups A and B with a 2-fold increase in tetanus antibody titers measured 4 weeks after the immunization compared with prevaccination levels for subjects with prevaccination tetanus antibody titers ≧0.1 IU/mL, or with an antibody titer ≧0.2 IU/mL, measured 4 weeks after immunization for subjects with prevaccination tetanus antibody titers <0.1 IU/mL. Prevaccination levels were those obtained immediately prior to administration of a vaccine.
2. The proportion of subjects in Groups A and B with positive responses against an individual anti-pneumococcal antibody serotype measured 4 weeks after the 23-valent pneumococcal polysaccharide vaccine. A positive response against a serotype is defined as a 2-fold increase or an increase of >1 μg/mL from prevaccination levels. Prevaccination levels are those obtained immediately prior to receipt of a vaccine.
3. The proportion of subjects in Groups A and B with positive responses against at least 50% of the serotypes (≧6 out of 12) measured 4 weeks after the 23-valent pneumococcal polysaccharide vaccine.
4. The proportion of subjects in Groups A and B with a positive response against at least k (for k=1, 2, 3, 4, 5) out of 12 pneumococcal antibody serotypes.
5. Levels of anti-tetanus antibody in subjects in Groups A and B measured immediately prior to and 4 weeks after a booster vaccination.
6. Levels of anti-pneumococcal antibody in subjects in Groups A and B measured immediately prior to and 4 weeks after vaccination.
7. Levels of anti-KLH antibody in subjects in Groups A and B measured immediately prior to the first administration of KLH and 4 weeks after the first administration of KLH.
8. The proportion of subjects who maintain a positive response to Candida albicans from Day 1 to 24 weeks (Group A) and Day 1 to 12 weeks (Group B). A positive response for Candida albicans skin test is defined as >5 mm in the diameter of induration.

For endpoints 1, 2, 3, 4, and 8, the proportion of subjects in Group A with positive responses were compared with the proportion of subjects in Group B with positive responses.

For endpoints 5, 6, and 7, geometric means and standard deviations of the concentrations of IgG antibody measured prior to and 4 weeks after the booster vaccinations were calculated for Groups A and B.

Antibody concentrations below the lower limit of assay detection were assigned half the lower limit for calculation of geometric means.

### Analysis Populations, Disposition, and Patient Characteristics

The disposition and analysis populations of this study are shown in the following table:

| | RTX + MTX | MTX |
|---|---|---|
| Randomized, N = 103 | 69 | 34 |
| Completed 1° Study Periods (RTX: Wk 36, control: | 62 | 27 |
| Wk 12) N, % | | |
| Tetanus Eval Population*, N = 90 | 64 | 26 |
| 23PPV Eval Population*, N = 91 | 63 | 28 |
| KLH Eval Population*, N = 91 | 64 | 27 |
| C. Albicans Skin Test Eval Population*, N = 92 | 64 | 28 |

*Pts with vaccine admin and pre-and post vaccine titers and skin test within protocol-defined windows

Patient demographics (Tetanus Response-Evaluable Population) are provided below:

| | RTX + MTX | MTX |
|---|---|---|
| N = 90 | N = 64 | N = 26 |
| Female (%) | 73 | 77 |
| Age (years) Mean | 50.3 | 50.4 |
| Caucasian (%) | 70 | 81 |
| Weight (kg) Mean | 84.4 | 90.8 |

The following table provides patient demographics (Tetanus Response-Evaluable population):

| | RTX + MTX | MTX |
|---|---|---|
| N = 90 | N = 64 | N = 26 |
| Disease duration (yrs) | 8.8 | 8.2 |
| No of Previous DMARDS (Excl | 3.1 | 2.3 |
| MTX, incl anti-TNF), mean | | |
| Anti-TNF-naïve, % | 57.8 | 57.7 |
| Baseline MTX dose (mg), mean | 17.3 | 16.8 |
| Baseline Steroid Use, % | 42 | 19 |
| Baseline background Steroid Dose | 6.4 | 8.5 |
| (prednisone equivalent mg/day), | | |
| mean | | |
| Anti-CCP+, % | 64 | 65 |
| RF+, IU/ml, % | 67 | 77 |
| Baseline C. albicans Anergy, % | 52 | 29 |

### Immune Response Results

Peripheral CD19+ depletion during immunization for Group A patients (Active group) is shown in the following table. The majority of patients (92%) were B-cell depleted at the time of tetanus vaccination.

| | CD19 < LLN (80 cells/μl) |
|---|---|
| Baseline (pre-rituximab) | 4/65 (6.2%) |
| Week 24 | 60/65 (92.3%) |
| Week 28 | 56/63 (88.9%) |
| Week 36 | 42/55 (76.4%) |

Tetanus toxoid adsorbed vaccine responses are summarized below.

| | RTX + MTX | MTX | Difference |
|---|---|---|---|
| | N = 64 | N = 26 | (95% CI) |
| 4-fold Titer Increase* | 25 (39.1%) | 11 (42.3%) | −3.2% |
| (Primary EP) | | | (−25.7%, 19.2%) |
| 2- fold Titer Increase* | 35 (54.7%) | 16 (61.5%) | −6.9% |
| (Secondary EP) | | | (−29.2%, 15.5%) |

*or ≧0.2 IU/mL if prevacc titer <0.1 IU/mL: 6 pts with prevacc titer <0.1 IU/mL(3 RTX, 3 control);
2 with positive response (both RTX group)

Positive responses to 23-valent pneumococcal polysaccharide vaccine, and to at least 1, 2, 3, 4, 5, or 6 of 12 pneumococcal antibody serotypes are shown in FIGS. 3 and 4, respectively.

Keyhole limpet hemocyanin post vaccine titers were as follows:

| | RTX + MTX | MTX |
|---|---|---|
| N = 91 | N = 64 | N = 27 |
| Patients with detectable levels of | 30(46.9%) | 25(92.6%) |
| anti-KLH IgG 4 wks post vaccine | | |
| 4 weeks Post vaccine GMT | 539.5 | 1585.5 |
| 95% CI of GMT | 461.54, 630.61 | 1065.15, 2360.7 |
| Post vaccine Median Titer | 483.1 | 1617.0 |

The DTH response is a skin test which measures maintenance of T-cell memory immune response. The C. Albicans skin test data were:

| | RTX + MTX | MTX |
|---|---|---|
| N = 92 | n = 64 | N = 28 |
| Positive DTH response* | 31 (48.4%) | 20 (71.4%) |
| at Baseline | | |
| Maintain a positive DTH | 24 (77.4%) | 14 (70.0%) |
| response | | |

| | |
|---|---|
| Difference (95% CI) | 7.4% (−17.5%, 32.3%) |

*≧5 mm in duration

### SUMMARY

### Vaccine Responses

Response to a recall antigen, tetanus toxoid vaccine, appears to be conserved in rituximab-treated as compared with MTX only treated RA patients.
Ability to maintain DTH to C. Albicans skin test, a T-cell mediated response, appears to be conserved in rituximab-treated as compared with MTX only treated RA patients.
Responses to 23-valent pneumococcal polysaccharide vaccine, a T-cell independent response, and to KLH, a neoantigen, appear decreased in rituximab-treated as compared with MTX only treated RA patients.

### Safety

The safety profile of rituximab-treated RA patients is consistent with previous experience with rituximab.

### CONCLUSIONS

Recall responses appear preserved in RTX-treated RA patients.
While neoantigen and T-cell independent responses appear decreased in more RTX-treated than in MTX only treated RA patients, many patients are able to mount responses. Therefore, immunization with such vaccines is recommended.
The inability of CD20 to abrogate all humoral vaccine responses may be due to adequate reserve in B-cell niche compartments not affected by CD20, and start of repletion at time of vaccination
RA patients treated with RTX can receive non-live vaccines.
Primary immunization with non-live vaccines should be considered prior to RTX infusion in RA patients, if possible.

While these data concern the rituximab CD20 antibody, based on these data it is contemplated that, other CD20 antibodies, specifically including humanized 2H7, ofatumumab, veltuzumab, TRU-015, AME 133v, and GA101, will result in a similar immune response to protein and/or polysaccharide vaccinations, particularly where such antibodies, like rituximab, have the biological functions of: antibody-dependent cellular cytotoxicity (ADCC), complement-dependent cytotoxicity (CDC), inducing apoptosis, and/or depleting B-cells.

What is claimed is:

1. A method of treating a human rheumatoid arthritis (RA) patient comprising administering to the patient: (a) a CD20 antibody in an amount effective to treat the RA, and (b) a protein vaccine in an amount effective to mount a memory immune response to the protein vaccine.

2. The method of claim 1 wherein the protein vaccine is a tetanus toxoid vaccine.

3. The method of claim 1 wherein the protein vaccine is administered to the patient following administration of the CD20 antibody.

4. The method of claim 3 wherein the protein vaccine is administered about six months after administration of the CD20 antibody.

5. The method of claim 1 wherein the patient has a 4-fold increase in anti-protein titer following administration of the protein vaccine.

6. The method of claim 2 wherein the patient has a 4-fold increase in anti-tetanus titer following administration of the tetanus toxoid vaccine.

7. The method of claim 1 further comprising administering methotrexate to the patient in an amount effective to treat the RA.

8. The method of claim 7 wherein the memory response to the vaccine is about the same as that mounted in a patient treated with methotrexate without the CD20 antibody.

9. The method of claim 1 further comprising eliciting a delayed-type hypersensitivity (DTH) response by administering to the patient an antigen which results in a T-cell mediated response in an amount effective to generate the DTH response.

10. The method of claim 9 wherein the antigen which generates the DTH response is C. Albicans.

11. The method of claim 1 further comprising administering a polysaccharide vaccine to the patient in an amount effective to mount an immune response to the polysaccharide vaccine.

12. The method of claim 11 wherein the polysaccharide vaccine is pneumococcal polysaccharide vaccine.

13. The method of claim 1 further comprising administering a neoantigen vaccine to the patient in an amount effective to mount a primary humoral immune response to the neoantigen vaccine.

14. The method of claim 13 wherein the neoantigen is Keyhole Limpet Hemocyanin (KLH).

15. The method of claim 1 wherein the CD20 antibody is selected from the group consisting of a chimeric, humanized, and human CD20 antibody.

16. The method of claim 1 wherein the CD20 antibody is rituximab.

17. The method of claim 1 wherein the CD20 antibody is humanized 2H7.

18. The method of claim 1 wherein the CD20 antibody is ofatumumab.

19. The method of claim 1 wherein the CD20 antibody is veltuzumab.

20. The method of claim 1 wherein the CD20 antibody is TRU-015.

21. The method of claim 1 wherein the CD20 antibody is GA 101.

22. The method of claim 1 wherein the CD20 antibody is AME-133v.

23. A method of mounting a 4-fold increase in anti-protein titer in a human rheumatoid arthritis (RA) patient following vaccination with a protein vaccine, comprising administering the protein vaccine to a RA patient who has been treated with a CD20 antibody, and measuring the 4-fold increase in anti-protein titer in the patient.

24. A method of treating human patients having rheumatoid arthritis (RA) comprising treating a first group of the RA patients with a CD20 antibody, methotrexate, and a protein vaccine, and treating a second group of the RA patients with methotrexate and the protein vaccine but not the CD20 antibody, and determining that memory immune responses raised by the first and second groups of patients are about the same.

25. The method of claim 24 wherein the protein vaccine is tetanus toxoid and the immune response is 4-fold increase in anti-tetanus titer following vaccination with the tetanus toxoid vaccine.

26. The method of claim 24 wherein the first group of patients includes at least 50 patients.

27. A method for advertising a CD20 antibody comprising promoting the use of the CD20 antibody for treating a human rheumatoid arthritis (RA) patient, wherein the RA patient is able to mount an effective memory immune response to a protein vaccine administered following administration of the CD20 antibody.

28. The method of claim 27 wherein the protein vaccine is a tetanus toxoid vaccine.

29. The method of claim 27 wherein the effective memory immune response is selected from the group consisting of a 2-fold rise in anti-tetanus titer and 4-fold rise in anti-tetanus titer.

30. The method of claim 27 wherein the patient is further able to mount an immune response to a polysaccharide vaccine or to a neoantigen vaccine.

31. The method of claim 30 wherein the polysaccharide vaccine is pneumococcal polysaccharide vaccine and the neoantigen vaccine is Keyhole Limpet Hemocyanin (KLH).

32. A method of providing a pharmaceutical composition for treating a human rheumatoid arthritis (RA) patient, comprising combining a container holding a pharmaceutically acceptable composition comprising a CD20 antibody with a package insert, wherein the package insert promotes the use of the composition to treat a RA patient who is able to mount an effective memory immune response to a protein vaccine administered following administration of the CD20 antibody.


SEQUENCE LISTING (18 sequences)

SEQ ID NO: 1 - protein, 107 aa, Artificial sequence
  Sequence is synthesized.
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 2 - protein, 122 aa, Artificial sequence [sequence omitted]

SEQ ID NO: 3 - protein, 107 aa, Artificial sequence
  Sequence is synthesized.
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 4 - protein, 122 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 5 - protein, 107 aa, Artificial sequence; identical to SEQ ID NO: 3
  Sequence is synthesized.

SEQ ID NO: 6 - protein, 122 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 7 - protein, 213 aa, Artificial sequence
  Sequence is synthesized.
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 451 aa, Artificial sequence
  Sequence is synthesized.
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 451 aa, Artificial sequence
  Sequence is synthesized.
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 213 aa, Artificial sequence
  Sequence is synthesized.
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 451 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 451 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCAVSNKALPAPIEATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 451 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 451 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 15 - protein, 451 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 451 aa, Artificial sequence
  Sequence is synthesized.
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 106 aa, Artificial sequence [sequence omitted]

SEQ ID NO: 18 - protein, 121 aa, Artificial sequence
  Sequence is synthesized.
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVS